(19) United States
(12) Patent Application Publication
(10) Pub. No.: US 2021/0388015 A1
(43) Pub. Date: Dec. 16, 2021

(54) GLUCOPYRANOSE DERIVATIVES USEFUL AS SGLT2 INHIBITORS

(71) Applicant: Janssen Pharmaceutica NV, Beerse (BE)

(72) Inventor: Guozhang Xu, Chesterbrook, PA (US)

(21) Appl. No.: 17/288,575

(22) PCT Filed: Oct. 24, 2019
(86) PCT No.: PCT/IB2019/059123

§ 371 Date: Apr. 26, 2021

Related U.S. Application Data
(60) Provisional application No. 62/750,864, filed on Oct. 26, 2018.

(51) Int. Cl. C07H 7/04; C07H 7/06

(52) U.S. Cl. CPC C07H 7/04; C07H 7/06

(57) ABSTRACT

The present invention is directed to glucopyranose derivatives, pharmaceutical compositions containing them and their use in the treatment of disorders and conditions modulated by SGLT2 activity. More particularly, the compounds of the present invention are useful in the treatment of for example, Type II diabetes mellitus, Syndrome X, and complications and symptoms associated with said disorders.

### CROSS REFERENCE TO RELATED APPLICATIONS

This Application claims priority to U.S. Provisional Patent Application No. 62/750,864, filed Oct. 26, 2018, the disclosure of which is hereby incorporated by reference in its entirety.

### FIELD OF THE INVENTION

The present invention is directed to glucopyranose derivatives, pharmaceutical compositions containing them and their use in the treatment of disorders and conditions modulated by SGLT2 activity. More particularly, the compounds of the present invention are useful in the treatment of for example, Type II diabetes mellitus, Syndrome X, and complications and symptoms associated with said disorders.

### BACKGROUND OF THE INVENTION

Diabetes is a chronic disorder affecting carbohydrate, fat and protein metabolism in animals.

Type I diabetes mellitus, which comprises approximately 10% of all diabetes cases, was previously referred to as insulin-dependent diabetes mellitus (IDDM) or juvenile onset diabetes. This disease is characterized by a progressive loss of insulin secretory function by beta cells of the pancreas. This characteristic is also shared by non-idiopathic, or “secondary”, diabetes having its origins in pancreatic disease. Type I diabetes mellitus is associated with the following clinical signs or symptoms: persistently elevated plasma glucose concentration or hyperglycemia; polyuria; polydipsia and/or hyperphagia; chronic microvascular complications such as retinopathy, nephropathy and neuropathy; and macrovascular complications such as hyperlipidemia and hypertension which can lead to blindness, end-stage renal disease, limb amputation and myocardial infarction. Therapy for IDDM patients has consistently focused on administration of exogenous insulin, which may be derived from various sources (e.g., human, bovine, porcine insulin). The use of heterologous species material gives rise to formation of anti-insulin antibodies which have activity limiting effects and result in progressive requirements for larger doses in order to achieve desired hypoglycemic effects.

Type II diabetes mellitus (non-insulin-dependent diabetes mellitus or NIDDM) is a metabolic disorder involving the dysregulation of glucose metabolism and impaired insulin sensitivity. Type II diabetes mellitus usually develops in adulthood and is associated with the body's inability to utilize or make sufficient insulin. In addition to the insulin resistance observed in the target tissues, patients suffering from Type II diabetes mellitus have a relative insulin deficiency—that is, patients have lower than predicted insulin levels for a given plasma glucose concentration. Type II diabetes mellitus is characterized by the following clinical signs or symptoms: persistently elevated plasma glucose concentration or hyperglycemia; polyuria; polydipsia and/or hyperphagia; chronic microvascular complications such as retinopathy, nephropathy and neuropathy; and macrovascular complications such as hyperlipidemia and hypertension which can lead to blindness, end-stage renal disease, limb amputation and myocardial infarction. Typical treatment of Type II diabetes mellitus focuses on maintaining the blood glucose level as near to normal as possible with lifestyle modification relating to diet and exercise, and when necessary, the treatment with antidiabetic agents, insulin or a combination thereof. NIDDM that cannot be controlled by dietary management is treated with oral antidiabetic agents.

Syndrome X, also termed Insulin Resistance Syndrome (IRS), Metabolic Syndrome, or Metabolic Syndrome X, is recognized in some 2% of diagnostic coronary catheterizations. Often disabling, it presents symptoms or risk factors for the development of Type II diabetes mellitus and cardiovascular disease, including impaired glucose tolerance (IGT), impaired fasting glucose (IFG), hyperinsulinemia, insulin resistance, dyslipidemia (e.g., high triglycerides, low HDL), hypertension and obesity. Although insulin resistance is not always treated in all Syndrome X patients, those who exhibit a prediabetic state (e.g., IGT, IFG), where fasting glucose levels may be higher than normal but not at the diabetes diagnostic criterion, is treated in some countries (e.g., Germany) with metformin to prevent diabetes. The anti-diabetic agents may be combined with pharmacological agents for the treatment of the concomitant co-morbidities (e.g., antihypertensives for hypertension, hypolipidemic agents for lipidemia).

Hyperglycemia is one common characteristic of these diabetic disorders. Treatments of hyperglycemia are focused on excretion of excessive glucose directly into urine, which involves sodium-glucose cotransporters (SGLTs), primarily found in the chorionic membrane of the intestine and kidney. In particular, renal reabsorption of glucose is mediated by SGLT1 and SGLT2 (MARSENIC, O., “Glucose Control by the Kidney: An Emerging Target in Diabetes”, AM. J. Kidney Dis., 2009 May, pp 875-883, Vol. 53(5); WRIGHT, E. M., et al., “Biology of Human Sodium Glucose Transporters”, Physiol. Rev., 2011 April, pp 733-794, Vol. 91(2)). SGLT1, a high-affinity low-capacity transporter with a Na⁺:glucose transport ratio of 2:1, is present in intestinal and renal epithelial cells (LEE, W. S., et al., “The High Affinity Na+/Glucose Cotransporter. Re-evaluation of Function and Distribution of Expression”, J. Biol. Chem., 1994 Apr. 22, pp 12032-12039, Vol. 269(16)). On the other hand, SGLT2, also known as SAAT1, a low-affinity high-capacity transporter with a Na⁺:glucose transport ratio of 1:1, is found in the epithelium of the kidney (YOU, G., et al., “Molecular Characteristic of Na(+)-coupled Glucose Transporters in Adult and Embryonic Rat Kidney”, J. Biol. Chem., 1995 Dec. 8, pp 29365-29371, Vol. 270(49); CHEN, J., et al., “Quantitative PCR Tissue Expression Profiling of the Human SGLT2 Gene and Related Family Members”, Diabetes Ther., 2010 December, pp 57-92, Vol. 1(2)). In addition, glucose absorption in the intestine is primarily mediated by SGLT1 and SGLT2. Thus, inhibition of SGLT1 and SGLT2 reduces plasma glucose through suppression of glucose reabsorption in the kidney, which was demonstrated in rodent models of IDDM and NIDDM by increasing the excretion of glucose in urine and lowering blood glucose levels.

Non-alcoholic fatty liver disease (NAFLD) is one cause of a fatty liver, occurring when fat is deposited (steatosis) in the liver. NAFLD is considered to cover a spectrum of disease activity. This spectrum begins as fatty accumulation in the liver (hepatic steatosis). A liver can remain fatty without disturbing liver function, but by varying mechanisms and possible insults to the liver may also progress to become NASH, a state in which steatosis is combined with inflammation and fibrosis. Non-alcoholic steatohepatitis (NASH) is a progressive, severe form of NAFLD. Over a 10-year period, up to 20% of patients with NASH will develop cirrhosis of the liver, and 10% will suffer death related to liver disease. The exact cause of NAFLD is still unknown, however, both obesity and insulin resistance are thought to play a strong role in the disease process. The exact reasons and mechanisms by which the disease progresses from one stage to the next are not known.

NAFLD has been linked to insulin resistance (IR) and the metabolic syndrome (MS). As the renin-angiotensin system (RAS) plays a central role in insulin resistance, and subsequently in NAFLD and NASH, an attempt to block the deleterious effects of RAS overexpression has been proposed a target for treatment. While many potential therapies tested in NASH target only the consequences of this condition, or try to “get rid” of excessive fat, angiotensin receptor blockers (ARBs) may act as a tool for correction of the various imbalances that act in harmony in NASH/NAFLD. Indeed, by inhibiting RAS the intracellular insulin signaling pathway may be improved, resulting in better control of adipose tissue proliferation and adipokine production, as well as more balanced local and systemic levels of various cytokines. At the same time, by controlling the local RAS in the liver fibrosis may be prevented and the cycle that links steatosis to necroinflammation slowed down. (GEORGESCU, E. F., in Advances in Therapy, 2008, pp 1141-1174, Vol. 25, Issue 11)

SCAFOGLIO, C., et al., in “Functional expression of sodium-glucose transporters in cancer”, PNAS, 2015, pp E41111-E4119, Vol 112(3), describe the role of sodium-dependent glucose transporters (SGLTs) in pancreatic and prostate adenocarcinomas, and their role in cancer cell survival. SGLT2 was found to be functionally expressed in pancreatic and prostate adenocarcinomas and further found to block glucose uptake and reduce tumor growth and survival in a xenograft model of pancreatic cancer, suggesting that SGLT2 inhibitors.

SONG, Y., et al., in PCT Publication WO2009/026537 A1, published Feb. 26, 2009 describe benzylbenzene derivatives having an inhibitory effect on sodium-dependent glucose cotransporter SGLT. LIOU, J., et al., in PCT Publication WO2010/022313 A2, published Feb. 25, 2010 describe processes for the preparation of complexes that are useful in purifying compounds having an inhibitory effect on sodium-dependent glucose cotransporter SGLT.

There remains a need for SGLT2 inhibitor compounds that have pharmacokinetic and pharmacodynamic properties suitable for use as human pharmaceuticals.

### SUMMARY OF THE INVENTION

The present invention is directed to compounds of formula (I)

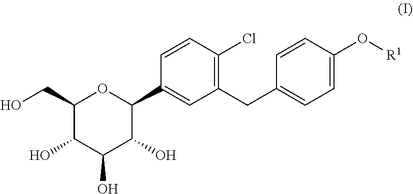

wherein

R¹ is selected from the group consisting of —CH₂CH₂—(OCH₂CH₂)ₐ—OCH₃, —CH₂CH₂CH₂—SO₃H, —(CH₂)_{b}—R², —CH₂CH₂—O—CH₂CH₂—R²,

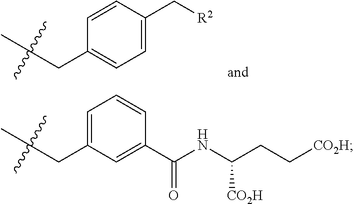

a is an integer from 2 to 12;

b is an integer from 2 to 6;

R² is selected from the group consisting of —N(CH₃)₃, 1-methyl-azetidin-1-yl, 1-methyl-pyrrolidin-1-yl, 1-methyl-piperidin-1-yl and 1-methyl-piperazin-1-yl;

and salts (preferably pharmaceutically acceptable salts) thereof.

The present invention is further directed to compounds of formula (II)

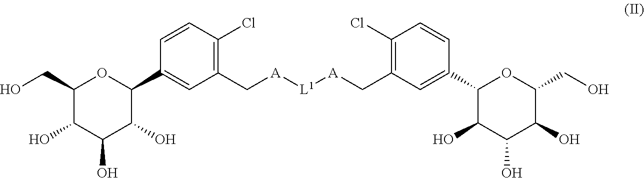

wherein each A is the same and is selected from the group consisting of

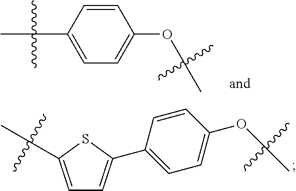

wherein the oxygen (O—) portion of the A group is bound to L¹;

L¹ is selected from the group consisting of —CH₂CH₂—(OCH₂CH₂)_{c}—, —CH(CO₂H)—CH₂CH₂—CH(CO₂H)—,

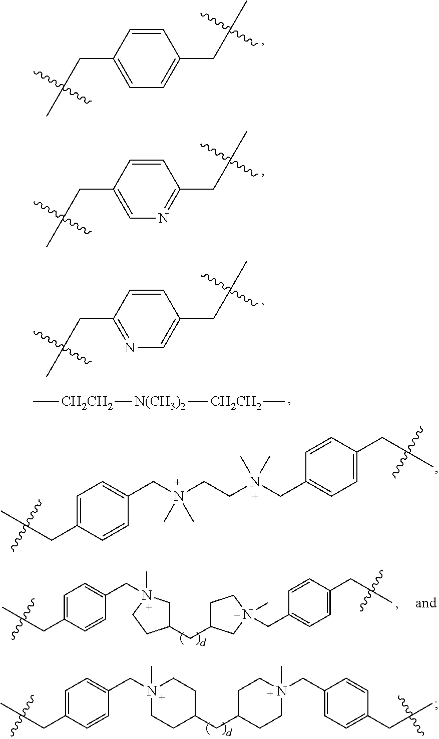

wherein c is an integer from 1 to 6;

wherein d is an integer from 0 to 2;

and salts (preferably pharmaceutically acceptable salts) thereof.

The present invention is further directed to a (tetramer) compound of formula (III)

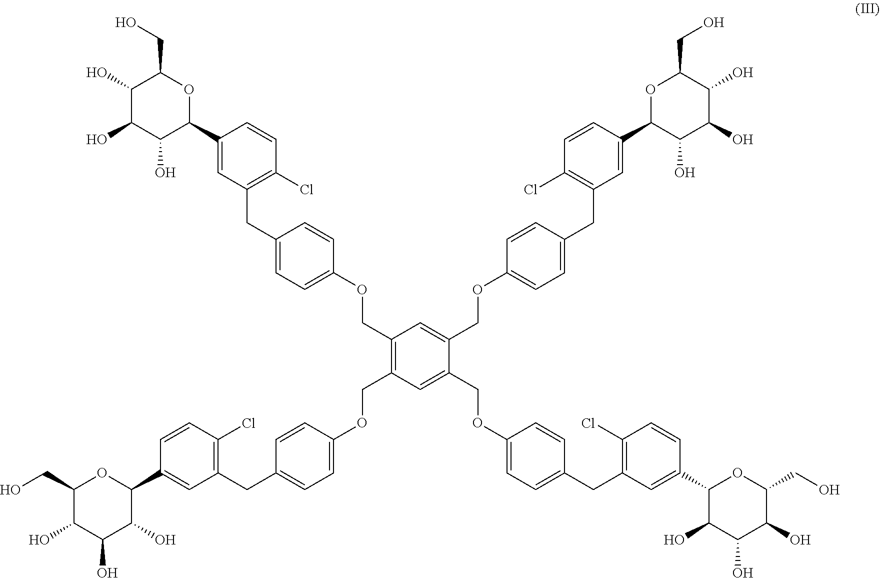 [SMILES: OC[C@@H]1O[C@H](c2ccc(Cl)c(Cc3ccc(OCc4cc(COc5ccc(Cc6cc([C@@H]7O[C@H](CO)[C@@H](O)[C@H](O)[C@H]7O)ccc6Cl)cc5)c(COc5ccc(Cc6cc([C@@H]7O[C@H](CO)[C@@H](O)[C@H](O)[C@H]7O)ccc6Cl)cc5)cc4COc4ccc(Cc5cc([C@@H]6O[C@H](CO)[C@@H](O)[C@H](O)[C@H]6O)ccc5Cl)cc4)cc3)c2)[C@@H](O)[C@H](O)[C@H]1O]

also known as (2R,2′R,2″R,3S,3′S,3″S,4R,4′R,4″R,5R,5′R,5″R,6S,6′S,6″S)-6,6′,6″-((((((5-((4-(2-chloro-5-((2R,3S,4S,5R,6S)-3,4,5-trihydroxy-6-(hydroxymethyl)tetrahydro-2H-pyran-2-yl)benzyl)phenoxy)methyl)benzene-1,2,4-triyl)tris(methylene))tris(oxy))tris(benzene-4,1-diyl))tris(methylene))tris(4-chlorobenzene-3,1-diyl))tris(2-(hydroxymethyl)tetrahydro-2H-pyran-3,4,5-triol), and salts (preferably pharmaceutically acceptable salts) thereof.

The present invention is further directed to processes for the preparation of the compounds of formula (I), formula (II) and formula (III). The present invention is further directed to a product prepared according to any of the process(es) described herein.

Illustrative of the invention are pharmaceutical compositions comprising a pharmaceutically acceptable carrier and a compound of formula (I), formula (II) or formula (III) as described herein. An illustration of the invention is a pharmaceutical composition made by mixing a compound of formula (I), formula (II) or formula (III) as described herein and a pharmaceutically acceptable carrier. Illustrating the invention is a process for making a pharmaceutical composition comprising mixing a compound of formula (I), formula (II) or formula (III) as described herein and a pharmaceutically acceptable carrier.

Exemplifying the invention are methods of treating a disease, disorder, or condition mediated by SGLT2 activity selected from the group consisting of impaired glucose tolerance (IGT), impaired fasting glucose (IFT), gestational diabetes, Type I diabetes mellitus, Type II diabetes mellitus, Syndrome X (also known as Metabolic Syndrome), obesity, nephropathy, neuropathy, retinopathy, hypertension, angina, atherosclerosis, heart disease, heart attack, ischemia, stroke, nerve damage or poor blood flow in the feet, non-alcoholic steatohepatitis (NASH), non-alcoholic fatty liver disease (NAFLD), liver fibrosis, cataracts, polycystic ovarian syndrome, irritable bowel syndrome, inflammation and cancer (preferably prostate cancer or pancreatic cancer), comprising administering to a subject in need thereof a therapeutically effective amount of any of the compounds or pharmaceutical compositions described above.

In an embodiment, the present invention is directed to a compound of formula (I), formula (II) or formula (III) for use as a medicament. In another embodiment, the present invention is directed to a compound of formula (I), formula (II) or formula (III) for use in the treatment of a disorder mediated SGLT2 selected from the group consisting impaired glucose tolerance (IGT), impaired fasting glucose (IFT), gestational diabetes, Type I diabetes mellitus, Type II diabetes mellitus, Syndrome X (also known as Metabolic Syndrome), obesity, nephropathy, neuropathy, retinopathy, hypertension, angina, atherosclerosis, heart disease, heart attack, ischemia, stroke, nerve damage or poor blood flow in the feet, non-alcoholic steatohepatitis (NASH), non-alcoholic fatty liver disease (NAFLD), liver fibrosis, cataracts, polycystic ovarian syndrome, irritable bowel syndrome, inflammation and cancer (preferably prostate cancer or pancreatic cancer). In another embodiment, the present invention is directed to a composition comprising a compound of formula (I), formula (II) or formula (III) for the treatment of a disorder mediated by SGLT2 selected from the group consisting impaired glucose tolerance (IGT), impaired fasting glucose (IFT), gestational diabetes, Type I diabetes mellitus, Type II diabetes mellitus, Syndrome X (also known as Metabolic Syndrome), obesity, nephropathy, neuropathy, retinopathy, hypertension, angina, atherosclerosis, heart disease, heart attack, ischemia, stroke, nerve damage or poor blood flow in the feet, non-alcoholic steatohepatitis (NASH), non-alcoholic fatty liver disease (NAFLD), liver fibrosis, cataracts, polycystic ovarian syndrome, irritable bowel syndrome, inflammation and cancer (preferably prostate cancer or pancreatic cancer).

Another example of the invention is the use of any of the compounds described herein in the preparation of a medicament for treating: (a) impaired glucose tolerance (IGT), (b) impaired fasting glucose (IFT), (c) gestational diabetes, (d) Type I diabetes mellitus, (e) Type II diabetes mellitus, (f) Syndrome X (also known as Metabolic Syndrome), (g) obesity, (h) nephropathy, (i) neuropathy, (j) retinopathy, (k) hypertension, (l) angina, (m) atherosclerosis, (n) heart disease, (o) heart attack, (p) ischemia, (q) stroke, (r) nerve damage or poor blood flow in the feet, (s) non-alcoholic steatohepatitis (NASH), (t) non-alcoholic fatty liver disease (NAFLD), (u) liver fibrosis, (v) cataracts, (w) polycystic ovarian syndrome, (x) irritable bowel syndrome, (y) inflammation and (z) cancer (preferably prostate cancer or pancreatic cancer), in a subject in need thereof. In another example, the present invention is directed to a compound as described herein for use in a methods for treating a disorder selected from the group consisting of impaired glucose tolerance (IGT), impaired fasting glucose (IFT), gestational diabetes, Type I diabetes mellitus, Type II diabetes mellitus, Syndrome X (also known as Metabolic Syndrome), obesity, nephropathy, neuropathy, retinopathy, hypertension, angina, atherosclerosis, heart disease, heart attack, ischemia, stroke, nerve damage or poor blood flow in the feet, non-alcoholic steatohepatitis (NASH), non-alcoholic fatty liver disease (NAFLD), liver fibrosis, cataracts, polycystic ovarian syndrome, irritable bowel syndrome, inflammation and cancer (preferably prostate cancer or pancreatic cancer), in a subject in need thereof.

### DETAILED DESCRIPTION OF THE INVENTION

The present invention is directed to compounds of formula (I)

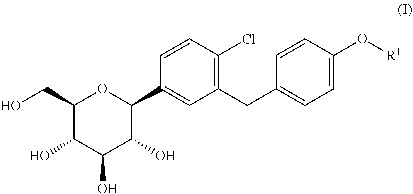

wherein R¹ is as herein defined; and salts, preferably pharmaceutically acceptable salts, thereof. The compounds of formula (I) of the present invention are useful in the treatment of diseases, disorders and complications associated with SGLT2 activity selected from the group of impaired glucose tolerance (IGT), impaired fasting glucose (IFT), gestational diabetes, Type I diabetes mellitus, Type II diabetes mellitus, Syndrome X (also known as Metabolic Syndrome), obesity, nephropathy, neuropathy, retinopathy, hypertension, angina, atherosclerosis, heart disease, heart attack, ischemia, stroke, nerve damage or poor blood flow in the feet, non-alcoholic steatohepatitis (NASH), non-alcoholic fatty liver disease (NAFLD), liver fibrosis, cataracts, polycystic ovarian syndrome, irritable bowel syndrome, inflammation and cancer (preferably prostate cancer or pancreatic cancer).

The present invention is further directed to compounds of formula (II)

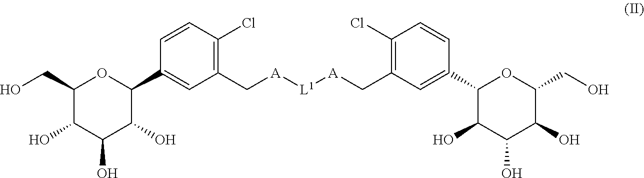

wherein A and L¹ are as herein defined; and salts, preferably pharmaceutically acceptable salts, thereof. The compounds of formula (II) of the present invention are useful in the treatment of diseases, disorders and complications associated with SGLT2 activity selected from the group of impaired glucose tolerance (IGT), impaired fasting glucose (IFT), gestational diabetes, Type I diabetes mellitus, Type II diabetes mellitus, Syndrome X (also known as Metabolic Syndrome), obesity, nephropathy, neuropathy, retinopathy, hypertension, angina, atherosclerosis, heart disease, heart attack, ischemia, stroke, nerve damage or poor blood flow in the feet, non-alcoholic steatohepatitis (NASH), non-alcoholic fatty liver disease (NAFLD), liver fibrosis, cataracts, polycystic ovarian syndrome, irritable bowel syndrome, inflammation and cancer (preferably prostate cancer or pancreatic cancer).

The present invention is further directed to a compound of formula (III)

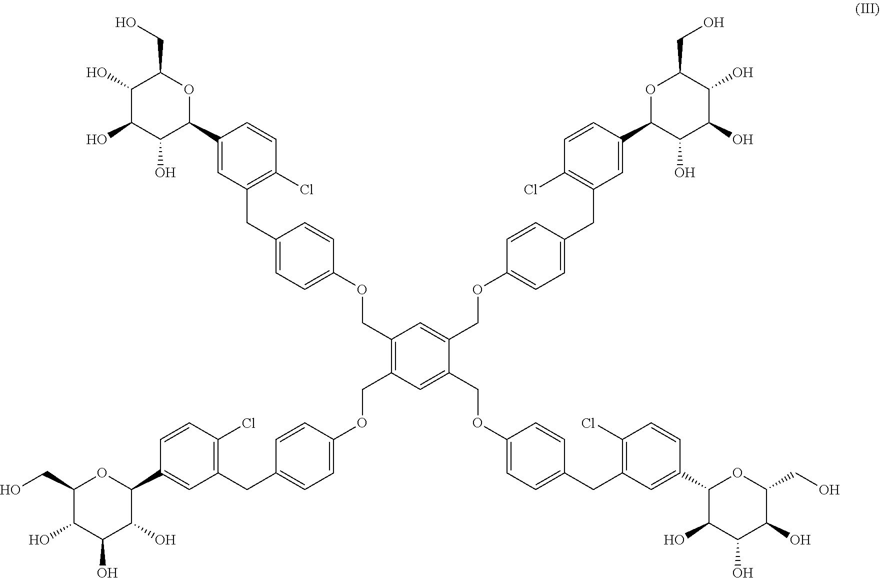 [SMILES: OC[C@@H]1O[C@H](c2ccc(Cl)c(Cc3ccc(OCc4cc(COc5ccc(Cc6cc([C@@H]7O[C@H](CO)[C@@H](O)[C@H](O)[C@H]7O)ccc6Cl)cc5)c(COc5ccc(Cc6cc([C@@H]7O[C@H](CO)[C@@H](O)[C@H](O)[C@H]7O)ccc6Cl)cc5)cc4COc4ccc(Cc5cc([C@@H]6O[C@H](CO)[C@@H](O)[C@H](O)[C@H]6O)ccc5Cl)cc4)cc3)c2)[C@@H](O)[C@H](O)[C@H]1O]

and salts, preferably pharmaceutically acceptable salts, thereof. The compound of formula (III) the present invention is useful in the treatment of diseases, disorders and complications associated with SGLT2 activity selected from the group of impaired glucose tolerance (IGT), impaired fasting glucose (IFT), gestational diabetes, Type I diabetes mellitus, Type II diabetes mellitus, Syndrome X (also known as Metabolic Syndrome), obesity, nephropathy, neuropathy, retinopathy, hypertension, angina, atherosclerosis, heart disease, heart attack, ischemia, stroke, nerve damage or poor blood flow in the feet, non-alcoholic steatohepatitis (NASH), non-alcoholic fatty liver disease (NAFLD), liver fibrosis, cataracts, polycystic ovarian syndrome, irritable bowel syndrome, inflammation and cancer (preferably prostate cancer or pancreatic cancer).

The compounds of formula (I), the compounds of formula (II) and the compound of formula (III) may alternatively be used in biological assays measuring SGLT2 activity, particularly SGLT2 activity associated with binding to extracellular membrane of renal proximal tubular cells.

In an embodiment, the present invention is directed to compounds of formula (I) wherein R¹ is selected from the group consisting of —CH₂CH₂—(OCH₂CH₂)ₐ—OCH₃, —CH₂CH₂CH₂—SO₃H, —(CH₂)_{b}—R², —CH₂CH₂—O—CH₂CH₂—R²,

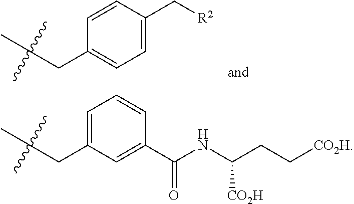

In an embodiment, the present invention is directed to compounds of formula (I) wherein a is an integer from 2 to 10. In another embodiment, the present invention is directed to compounds of formula (I) wherein a is an integer from 2 to 8. In another embodiment, the present invention is directed to compounds of formula (I) wherein a is an integer from 2 to 6. In another embodiment, the present invention is directed to compounds of formula (I) wherein a is an integer from 2 to 4. In another embodiment, the present invention is directed to compounds of formula (I) wherein a is 2, 3 or 4. In another embodiment, the present invention is directed to compounds of formula (I) wherein a is 3.

In another embodiment, the present invention is directed to compounds of formula (I) wherein R¹ is selected from the group consisting of —CH₂CH₂—(OCH₂CH₂)₃—OCH₃, —CH₂CH₂CH₂—SO₃H, —CH₂CH₂CH₂—N⁺(CH₃)₃,

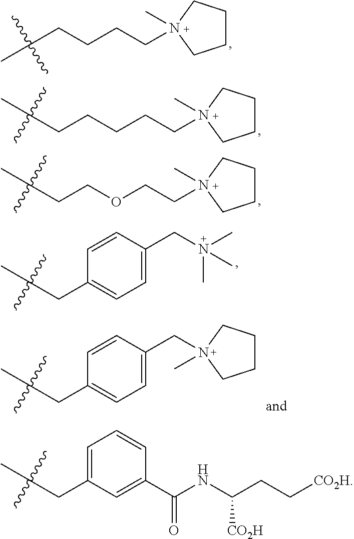

In another embodiment, the present invention is directed to compounds of formula (I) wherein R¹ is selected from the group consisting of —CH₂CH₂CH₂—

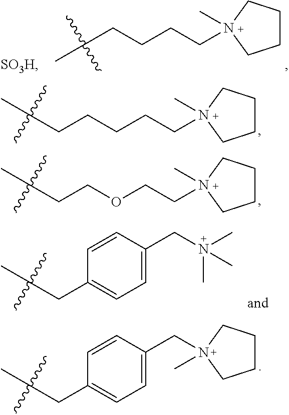

In another embodiment, the present invention is directed to compounds of formula (I) wherein R¹ is selected from the group consisting of

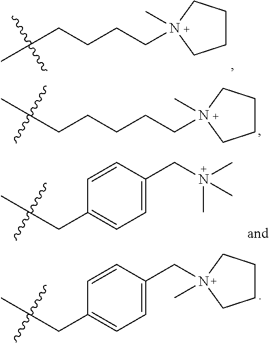

In an embodiment, the present invention is directed to compounds of formula (I) wherein b is an integer from 2 to 5. In another embodiment, the present invention is directed to compounds of formula (I) wherein b is an integer from 3 to 5. In another embodiment, the present invention is directed to compounds of formula (I) wherein b is selected from the group consisting of 2, 3, 4 and 5, preferably b is selected from the group consisting of 3, 4, and 5, more preferably, b is 4 or 5.

In an embodiment, the present invention is directed to compounds of formula (I) wherein R² is selected from the group consisting of —N(CH₃)₃, 1-methyl-azetidin-1-yl, 1-methyl-pyrrolidin-1-yl and 1-methyl-piperidin-1-yl.

In another embodiment, the present invention is directed to a compound of formula (I) selected from the group consisting of

1-(4-(4-(2-chloro-5-((2S,3R,4R,5S,6R)-3,4,5-trihydroxy-6-(hydroxymethyl)tetrahydro-2H-pyran-2-yl)benzyl)phenoxy)butyl)-1-methylpyrrolidin-1-ium trifluoroacetate;

1-(5-(4-(2-chloro-5-((2S,3R,4R,5S,6R)-3,4,5-trihydroxy-6-(hydroxymethyl)tetrahydro-2H-pyran-2-yl)benzyl)phenoxy)pentyl)-1-methylpyrrolidin-1-ium trifluoroacetate;

1-(4-((4-(2-chloro-5-((2S,3R,4R,5S,6R)-3,4,5-trihydroxy-6-(hydroxymethyl)tetrahydro-2H-pyran-2-yl)benzyl)phenoxy)methyl)phenyl)-N,N,N-trimethylmethanaminium trifluoroacetate; and

1-(4-((4-(2-chloro-5-((2S,3R,4R,5S,6R)-3,4,5-trihydroxy-6-(hydroxymethyl)tetrahydro-2H-pyran-2-yl)benzyl)phenoxy)methyl)benzyl)-1-methylpyrrolidin-1-ium trifluoroacetate.

In an embodiment, the present invention is directed to the trifluoroacetate salts of the compounds of formula (I).

In an embodiment, the present invention is directed to a compound of formula (II) wherein each A is the same and is

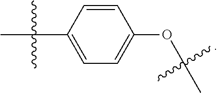 [SMILES: *Oc1ccc(*)cc1]

In another embodiment, the present invention is directed to a compound of formula (II) wherein each A is the same and is

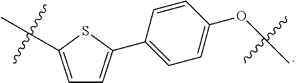 [SMILES: *Oc1ccc(-c2ccc(*)s2)cc1]

In an embodiment, the present invention is directed to compounds of formula (II) wherein L¹ is selected from the group consisting of —CH₂CH₂—(OCH₂CH₂)_{c}—, —CH(CO₂H)—CH₂CH₂—CH(CO₂H)—,

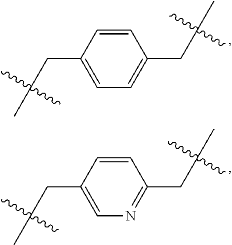 [SMILES: *Cc1ccc(C*)cc1 ; *Cc1ccc(C*)nc1]

—CH₂CH₂—N(CH₃)₂—CH₂CH₂—,

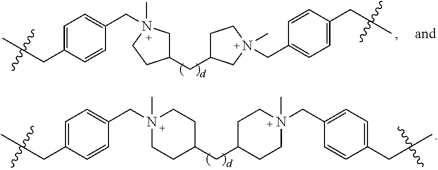

In another embodiment, the present invention is directed to compounds of formula (II) wherein L¹ is selected from the group consisting of —CH₂CH₂—O—CH₂CH₂—, —CH₂CH₂—O—CH₂CH₂—O—CH₂CH₂—, —CH₂CH₂—O—CH₂CH₂—O—CH₂CH₂—O—CH₂CH₂—, —CH(CO₂H)—CH₂CH₂—CH(CO₂H)—,

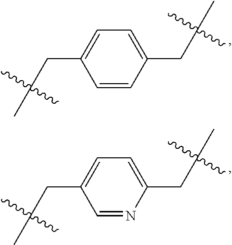 [SMILES: *Cc1ccc(C*)cc1 ; *Cc1ccc(C*)nc1]

—CH₂CH₂—N⁺(CH₃)₂—CH₂CH₂—,

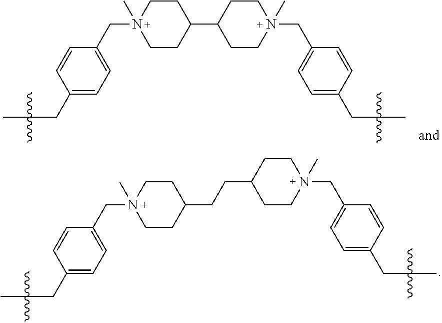

In another embodiment, the present invention is directed to compounds of formula (II) wherein L¹ is selected from the group consisting of —CH₂CH₂—O—CH₂CH₂—, —CH₂CH₂—O—CH₂CH₂—O—CH₂CH₂—, —CH₂CH₂—O—CH₂CH₂—O—CH₂CH₂—O—CH₂CH₂—,

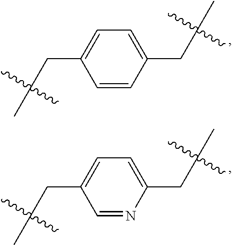 [SMILES: *Cc1ccc(C*)cc1 ; *Cc1ccc(C*)nc1]

—CH₂CH₂—N⁺(CH₃)₂—CH₂CH₂—,

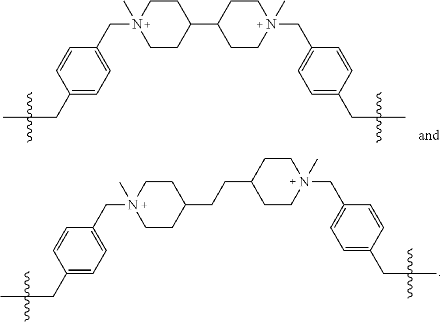

In another embodiment, the present invention is directed to compounds of formula (II) wherein L¹ is selected from the group consisting of —CH₂CH₂—O—CH₂CH₂—, —CH₂CH₂—O—CH₂CH₂—O—CH₂CH₂—O—CH₂CH₂—,

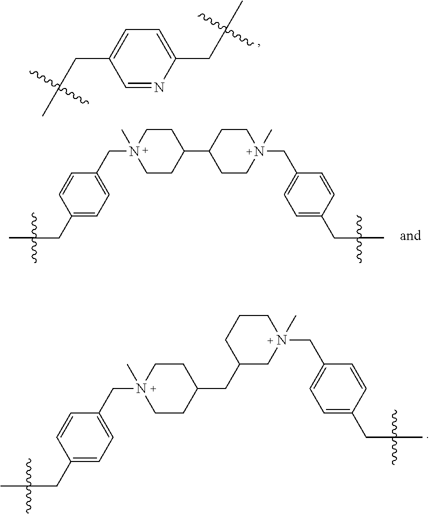

In an embodiment, the present invention is directed to compounds of formula (II) wherein c is an integer from 0 to 4. In another embodiment, the present invention is directed to compounds of formula (II) wherein c is an integer from 1 to 4. In another embodiment, the present invention is directed to compounds of formula (II) wherein c is an integer from 1 to 3. In another embodiment, the present invention is directed to compounds of formula (II) wherein c is 1. In another embodiment, the present invention is directed to compounds of formula (II) wherein c is 2. In another embodiment, the present invention is directed to compounds of formula (II) wherein c is 3.

In an embodiment, the present invention is directed to compounds of formula (II) wherein d is 0. In an embodiment, the present invention is directed to compounds of formula (II) wherein d is 1. In an embodiment, the present invention is directed to compounds of formula (II) wherein d is 2. In an embodiment, the present invention is directed to compounds of formula (II) wherein d is selected from the group consisting of 0 and 2.

In another embodiment, the present invention is directed to a compound of formula (II) selected from the group consisting of

(2R,2′R,3S,3′S,4R,4′R,5R,5′R,6S,6′S)-6,6′-(((((pyridine-2,5-diylbis(methylene))bis(oxy))bis(4,1-phenylene))bis(methylene))bis(4-chloro-3,1-phenylene))bis(2-(hydroxymethyl)tetrahydro-2H-pyran-3,4,5-triol) trifluoroacetate;

1,1′-bis(4-((4-(2-chloro-5-((2S,3R,4R,5S,6R)-3,4,5-trihydroxy-6-(hydroxymethyl)tetrahydro-2H-pyran-2-yl)benzyl)phenoxy)methyl)benzyl)-1,1′-dimethyl-[4,4′-bipiperidine]-1,1′-diium trifluoroacetate; and

4,4′-(ethane-1,2-diyl)bis(1-(4-((4-(2-chloro-5-((2S,3R,4R,5S,6R)-3,4,5-trihydroxy-6-(hydroxymethyl)tetrahydro-2H-pyran-2-yl)benzyl)phenoxy)methyl)benzyl)-1-methylpiperidin-1-ium) trifluoroacetate.

In an embodiment, the present invention is directed to the trifluoroacetate salts of the compounds of formula (II).

In an embodiment, the present invention is directed to a compound of formula (III) and salts thereof. In another embodiment, the present invention is directed to the trifluoroacetate salt of the compound of formula (III).

Additional embodiments of the present invention, include those wherein the substituents selected for one or more of the variables defined herein (i.e. R¹, A, L¹, etc.) are independently selected to be any individual substituent or any subset of substituents selected from the complete list as defined herein.

Additional embodiments of the present invention, include those wherein the substituents selected for one or more of the variables defined herein (i.e. R¹, A, L¹, etc.) are independently selected to correspond to any of the embodiments as defined herein.

In another embodiment of the present invention is any single compound or subset of compounds selected from the representative compounds listed in Tables 1 to 2 below.

Representative compounds of formula (I) and representative compounds of formula (II) of the present invention are as listed in Table 1 to 2, below. Wherein the compound of formula (I) or formula (II) contains a quaternary nitrogen, said compound was prepared as its corresponding salt, for example as its corresponding trifluoroacetate salt. Unless otherwise noted, wherein a stereogenic center is present in the listed compound, the compound was prepared as a mixture of stereo-configurations. Where a stereogenic center is present, the S*- and R* designations are intended to indicate that the exact stereo-configuration of the center has not been determined.

TABLE 1
Representative Compounds of Formula (I)

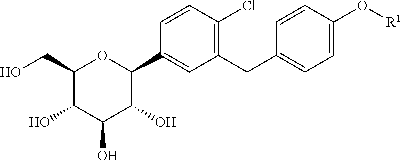

| Cmpd No. | R¹ |
|---|---|
| 1 | —CH₂CH₂—(OCH₂CH₂)₃—OCH₃ |
| 2 | —CH₂CH₂CH₂—SO₃H |
| 3 | 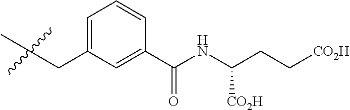 [SMILES: *Cc1cccc(C(=O)N[C@H](CCC(=O)O)C(=O)O)c1] |
| 4^{a} | —CH₂CH₂CH₂—N⁺(CH₃)₃ |
| 5^{a} | 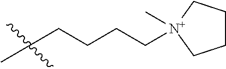 [SMILES: *CCCC[N+]1(C)CCCC1] |
| 6^{a} | 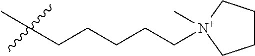 [SMILES: *CCCCC[N+]1(C)CCCC1] |
| 7^{a} | 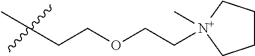 [SMILES: *CCOCC[N+]1(C)CCCC1] |
| 8^{a} | 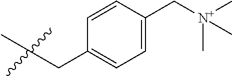 [SMILES: *Cc1ccc(C[N+](C)(C)C)cc1] |
| 9^{a} | 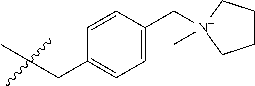 [SMILES: *Cc1ccc(C[N+]2(C)CCCC2)cc1] |

^{a}Compounds 4-9 were prepared as their corresponding TFA (CF₃COO⁻) salts.

TABLE 2
Representative Compounds of Formula (II)

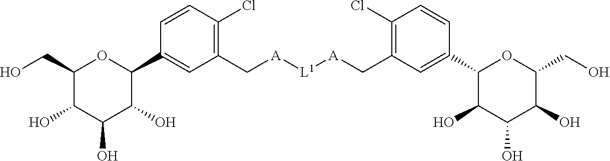

| Cmpd | | |
|---|---|---|
| No. | A | L¹ |
| 10 | 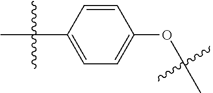 [SMILES: *Oc1ccc(*)cc1] | —CH₂CH₂—O—CH₂CH₂— |
| 11 | 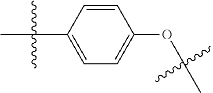 [SMILES: *Oc1ccc(*)cc1] | —CH₂CH₂—O—CH₂CH₂—O—CH₂CH₂— |
| 12 | 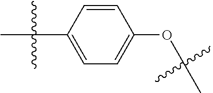 [SMILES: *Oc1ccc(*)cc1] | —CH₂CH₂—O—CH₂CH₂—O—CH₂CH₂—O—CH₂CH₂— |
| 13 | 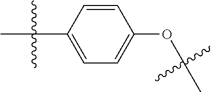 [SMILES: *Oc1ccc(*)cc1] | 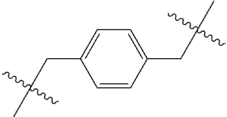 [SMILES: *Cc1ccc(C*)cc1] |
| 14 | 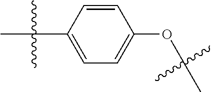 [SMILES: *Oc1ccc(*)cc1] | 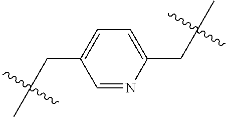 [SMILES: *Cc1ccc(C*)nc1] |
| 15^{b} | 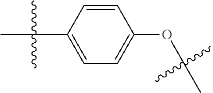 [SMILES: *Oc1ccc(*)cc1] | —CH₂CH₂—N⁺(CH₃)₂—CH₂CH₂— |
| 16^{c} | 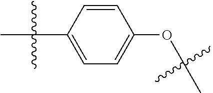 [SMILES: *Oc1ccc(*)cc1] | 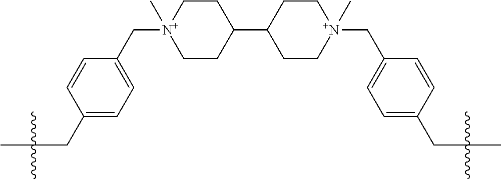 [SMILES: *Cc1ccc(C[N+]2(C)CCC(C3CC[N+](C)(Cc4ccc(C*)cc4)CC3)CC2)cc1] |
| 17^{c} | 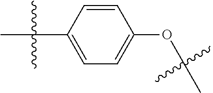 [SMILES: *Oc1ccc(*)cc1] | 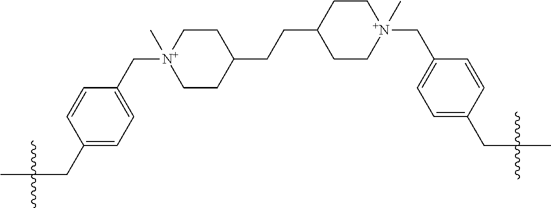 [SMILES: *Cc1ccc(C[N+]2(C)CCC(CCC3CC[N+](C)(Cc4ccc(C*)cc4)CC3)CC2)cc1] |
| 18 | 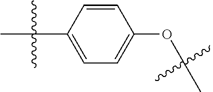 [SMILES: *Oc1ccc(*)cc1] | —CH(CO₂H)—CH₂CH₂—CH(CO₂H)— |
| 19^{b} | 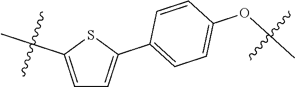 [SMILES: *Oc1ccc(-c2ccc(*)s2)cc1] | —CH₂CH₂—N⁺(CH₃)₂—CH₂CH₂— |

^{b}Compounds 15, 19 were prepared as corresponding TFA (CF₃COO⁻) salts.
^{c}Compounds 16, 17 were prepared as corresponding TFA (2•CF₃COO⁻) salts.
As used herein, the notation “*” shall denote the presence of a stereogenic center.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Preferably, wherein the compound is present as an enantiomer, the enantiomer is present at an enantiomeric excess of greater than or equal to about 80%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similarly, wherein the compound is present as a diastereomer, the diastereomer is present at an diastereomeric excess of greater than or equal to about 80%, more preferably, at an diastereomeric excess of greater than or equal to about 90%, more preferably still, at an diastereomeric excess of greater than or equal to about 95%, more preferably still, at an diastereomeric excess of greater than or equal to about 98%, most preferably, at an diastereomeric excess of greater than or equal to about 99%.

Furthermore, some of the crystalline forms for the compounds of the present invention may exist as polymorphs and as such are intended to be included in the present invention. In addition, some of the compounds of the present invention may form solvates with water (i.e., hydrates) or common organic solvents, and such solvates are also intended to be encompassed within the scope of this invention.

As used herein, unless otherwise noted, the term “isotopologues” shall mean molecules that differ only in their isotopic composition. More particularly, an isotopologue of a molecule differs from the parent molecule in that it contains at least one atom which is an isotope (i.e. has a different number of neutrons from its parent atom).

For example, isotopologues of water include, but are not limited to, “light water” (HOH or H₂O), “semi-heavy water” with the deuterium isotope in equal proportion to protium (HDO or ¹H²HO), “heavy water” with two deuterium isotopes of hydrogen per molecule (D₂O or ²H₂O), “super-heavy water” or tritiated water (T₂O or ³H₂O), where the hydrogen atoms are replaced with tritium (³H) isotopes, two heavy-oxygen water isotopologues (H₂¹⁸O and H₂¹⁷O) and isotopologues where the hydrogen and oxygen atoms may each independently be replaced by isotopes, for example the doubly labeled water isotopologue D₂¹⁸O.

It is intended that within the scope of the present invention, any one or more element(s), in particular when mentioned in relation to a compound of formula (I), shall comprise all isotopes and isotopic mixtures of said element(s), either naturally occurring or synthetically produced, either with natural abundance or in an isotopically enriched form. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O. The isotopes may be radioactive or non-radioactive. Radio-labeled compounds of formula (I) may comprise one or more radioactive isotope(s) selected from the group of ³H, ¹¹C, ¹⁸F, ¹²²I, ¹²³I, ¹²⁵I, ¹³¹I, ⁷⁵Br, ⁷⁶Br, ⁷⁷Br and ⁸²Br. Preferably, the radioactive isotope is selected from the group of ³H, ¹¹C and ¹⁸F.

Under standard nomenclature used throughout this disclosure, the terminal portion of the designated side chain is described first, followed by the adjacent functionality toward the point of attachment. Thus, for example, a “phenylC₁-C₆alkylaminocarbonylC₁-C₆alkyl” substituent refers to a group of the formula

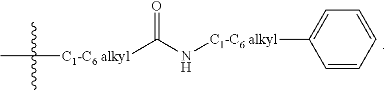

Abbreviations used in the specification, particularly the Schemes and Examples, are as listed in Table A, below.

TABLE A
Abbreviations

| | |
|---|---|
| Ac = | Acetyl (i.e —C(O)—CH₃) |
| AcCl = | Acetyl chloride |
| Al(OTf)₃ = | Aluminum tris(trifluoromethanesulfonate) |
| AMG = | ¹⁴C-α-methyl-d-glucopyranoside |
| ARB = | Angiotensin receptor blockers |
| aq. = | Aqueous |
| BF₃•Et₂O = | Boron trifluoride diethyl etherate |
| n-BuLi = | n-Butyl lithium |
| CHO K-1 (cells) = | Chinese hamster ovary K-1 (cells) |
| Cmpd = | Compound |
| DCM = | Dichloromethane |
| DIPEA or DIEA = | Diisopropylethylamine |
| DMAP = | 4-Dimethylaminopyridine |
| DMF = | N,N-Dimethylformamide |
| DMSO = | Dimethylsulfoxide |
| Et₂O = | Diethyl Ether |
| Et₃N or TEA = | Triethylamine |
| EtOAc or EA = | Ethyl acetate |
| EtOH = | Ethanol |
| HDL = | High density lipoprotein |
| HEPES = | 4-(2-Hydroxyethyl)-1-Piperizine Ethane Sulfonic |
| | Acid |
| HPLC = | High Pressure Liquid Chromatography |
| IDDM = | Insulin-dependent Diabetes Mellitus |
| IFG = | Impaired fasting glucose |
| IGT = | Impaired glucose tolerance |
| IRS = | Insulin resistance syndrome |
| LC/MS or LCMS = | Liquid chromatography-masss spectrometry |
| MDCK (cells) = | Madin-Darby Canine Kidney (cells) |
| Me = | Methyl (i.e. —CH₃) |
| MeI = | Methyl iodide |
| MeOH = | Methanol |
| MeSO₃H = | Methanesulfonic acid |
| MeSO₄ = | Methyl Sulfate |
| Mesyl or Ms = | Methylsulfonyl |
| 2-Methyl-THF = | 2-Methyl-tetrahydrofuran |
| MOM = | Methoxy methyl |
| MTBE = | Methyl t-butyl ether |
| NAFLD = | Non-alcoholic fatty liver disease |
| NASH = | Non-alcoholic steatohepatitis, |
| NaOMe = | Sodium methoxide |
| NBS = | N-Bromosuccinimide |
| NIDDM = | Non-insulin-dependent Diabetes Mellitus |
| NMR = | Nuclear magnetic resonance |
| OAc = | Acetoxy (i.e. —O—C(O)—CH₃) |
| OMe = | Methoxy |
| OTf = | Trifluoromethanesulfonate (triflate) |
| PdCl₂(dppf)₂ = | Palladium acetate |
| Pd₂dba₃ = | Tris(dibenzylideneacetone)dipalladium(0) |
| Pd(PPh₃)₄ = | Tetrakistriphenylphosphine palladium (0) |
| PyHBr₃ = | Pyridinium hydrobromide perbromide |
| i-PrMg•LiCl = | Isopropyl magnesium chloride/Lithium chloride |
| | complex |
| RAS = | Renin-angiotensin system |
| SGLT = | Sodium glucose transport |
| SGLT2 = | Sodium glucose transport-2 |
| TES = | Triethyl silane |
| TFA = | Trifluoroacetic Acid |
| TFAA = | Trifluoroacetic anhydride |
| TFE = | Trifluoroethylene |
| TfOH = | Trifluoromethanesulfonic acid (triflic acid) |
| THF = | Tetrahydrofuran |
| THP = | Tetrahydropyran |
| TLC = | Thin Layer Chromatography |
| TMS = | Trimethylsilyl |
| Tosyl or Ts = | p-Toluenesulfonyl |
| TsOH or p-TsOH = | p-Toluenesulfonic acid |
| Tris HCl or | Tris[hydroxymethyl]aminomethyl hydrochloride |
| Tris-Cl or | |
| Tris base = | |

As used herein, unless otherwise noted, the term “isolated form” shall mean that the compound is present in a form which is separate from any solid mixture with another compound(s), solvent system or biological environment. In an embodiment of the present invention, the compound of formula (I), formula (II) or formula (III) is present in an isolated form.

As used herein, unless otherwise noted, the term “substantially pure form” shall mean that the mole percent of impurities in the isolated compound is less than about 5 mole percent, preferably less than about 2 mole percent, more preferably, less than about 0.5 mole percent, most preferably, less than about 0.1 mole percent. In an embodiment of the present invention, the compound of formula (I), formula (II) or formula (III) is present as a substantially pure form.

As used herein, unless otherwise noted, the term “substantially free of a corresponding salt form(s)” when used to described the compound of formula (I), formula (II) or formula (III) shall mean that mole percent of the corresponding salt form(s) in the isolated base of formula (I), formula (II) or formula (III) is less than about 5 mole percent, preferably less than about 2 mole percent, more preferably, less than about 0.5 mole percent, most preferably less than about 0.1 mole percent. In an embodiment of the present invention, the compound of formula (I), formula (II) or formula (III) is present in a form which is substantially free of corresponding salt form(s).

As used herein, unless otherwise noted, the terms “treating”, “treatment” and the like, shall include the management and care of a subject or patient (preferably mammal, more preferably human) for the purpose of combating a disease, condition, or disorder and includes the administration of a compound of the present invention to prevent the onset of the symptoms or complications, to alleviate the symptoms or complications, to slow the progression of the disease or disorder, or to eliminate the disease, condition, or disorder.

As used herein, unless otherwise noted, the term “prevention” shall include (a) reduction in the frequency of one or more symptoms; (b) reduction in the severity of one or more symptoms; (c) the delay or avoidance of the development of additional symptoms; and/or (d) delay or avoidance of the development of the disorder or condition.

One skilled in the art will recognize that wherein the present invention is directed to methods of prevention, a subject in need of thereof (i.e. a subject in need of prevention) shall include any subject or patient (preferably a mammal, more preferably a human) who has experienced or exhibited at least one symptom of the disorder, disease or condition to be prevented. Further, a subject in need thereof may additionally be a subject (preferably a mammal, more preferably a human) who has not exhibited any symptoms of the disorder, disease or condition to be prevented, but who has been deemed by a physician, clinician or other medical profession to be at risk of developing said disorder, disease or condition. For example, the subject may be deemed at risk of developing a disorder, disease or condition (and therefore in need of prevention or preventive treatment) as a consequence of the subject's medical history, including, but not limited to, family history, pre-disposition, co-existing (comorbid) disorders or conditions, genetic testing, and the like.

The term “subject” as used herein, refers to an animal, preferably a mammal, most preferably a human, who has been the object of treatment, observation or experiment. Preferably, the subject has experienced and/or exhibited at least one symptom of the disease or disorder to be treated and/or prevented.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

As more extensively provided in this written description, terms such as “reacting” and “reacted” are used herein in reference to a chemical entity that is any one of: (a) the actually recited form of such chemical entity, and (b) any of the forms of such chemical entity in the medium in which the compound is being considered when named.

One skilled in the art will recognize that, where not otherwise specified, the reaction step(s) is performed under suitable conditions, according to known methods, to provide the desired product. One skilled in the art will further recognize that, in the specification and claims as presented herein, wherein a reagent or reagent class/type (e.g. base, solvent, etc.) is recited in more than one step of a process, the individual reagents are independently selected for each reaction step and may be the same of different from each other. For example wherein two steps of a process recite an organic or inorganic base as a reagent, the organic or inorganic base selected for the first step may be the same or different than the organic or inorganic base of the second step. Further, one skilled in the art will recognize that wherein a reaction step of the present invention may be carried out in a variety of solvents or solvent systems, said reaction step may also be carried out in a mixture of the suitable solvents or solvent systems.

One skilled in the art will recognize that wherein a reaction step of the present invention may be carried out in a variety of solvents or solvent systems, said reaction step may also be carried out in a mixture of the suitable solvents or solvent systems.

One skilled in the art will further recognize that the reaction or process step(s) as herein described are allowed to proceed for a sufficient period of time until the reaction is complete, as determined by any method known to one skilled in the art, for example, chromatography (e.g. HPLC). In this context a “completed reaction or process step” shall mean that the reaction mixture contains a significantly diminished amount of the starting material(s)/reagent(s) and a significantly reduced amount of the desired product(s), as compared to the amounts of each present at the beginning of the reaction.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any amount or range therein.

Examples of suitable solvents, bases, reaction temperatures, and other reaction parameters and components are provided in the detailed descriptions which follow herein. One skilled in the art will recognize that the listing of said examples is not intended, and should not be construed, as limiting in any way the invention set forth in the claims which follow thereafter.

As used herein, unless otherwise noted, the term “aprotic solvent” shall mean any solvent that does not yield a proton. Suitable examples include, but are not limited to DMF, 1,4-dioxane, THF, acetonitrile, pyridine, dichloroethane, dichloromethane, MTBE, toluene, acetone, and the like.

As used herein, unless otherwise noted, the term “leaving group” shall mean a charged or uncharged atom or group which departs during a substitution or displacement reaction. Suitable examples include, but are not limited to, Br, Cl, I, mesylate, tosylate, and the like.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

As used herein, unless otherwise noted, the term “nitrogen protecting group” shall mean a group which may be attached to a nitrogen atom to protect said nitrogen atom from participating in a reaction and which may be readily removed following the reaction. Suitable nitrogen protecting groups include, but are not limited to carbamates—groups of the formula —C(O)O—R wherein R is for example methyl, ethyl, t-butyl, benzyl, phenylethyl, CH₂═CH—CH₂—, and the like; amides—groups of the formula —C(O)—R′ wherein R′ is for example methyl, phenyl, trifluoromethyl, and the like; N-sulfonyl derivatives—groups of the formula —SO₂—R″ wherein R″ is for example tolyl, phenyl, trifluoromethyl, 2,2,5,7,8-pentamethylchroman-6-yl-, 2,3,6-trimethyl-4-methoxybenzene, and the like. Other suitable nitrogen protecting groups may be found in texts such as T. W. Greene & P. G. M. Wuts, Protective Groups in Organic Synthesis, John Wiley & Sons, 1991.

As used herein, unless otherwise noted, the term “oxygen protecting group” shall mean a group which may be attached to an oxygen atom to protect said oxygen atom from participating in a reaction and which may be readily removed following the reaction. Suitable oxygen protecting groups include, but are not limited to, acetyl, benzoyl, t-butyl-dimethylsilyl, trimethylsilyl (TMS), MOM, THP, and the like. Other suitable oxygen protecting groups may be found in texts such as T. W. Greene & P. G. M. Wuts, Protective Groups in Organic Synthesis, John Wiley & Sons, 1991.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

Additionally, chiral HPLC against a standard may be used to determine percent enantiomeric excess (% ee). The enantiomeric excess may be calculated as follows

[(Rmoles−Smoles)/(Rmoles+Smoles)]×100%

where Rmoles and Smoles are the R and S mole fractions in the mixture such that Rmoles+Smoles=1. The enantiomeric excess may alternatively be calculated from the specific rotations of the desired enantiomer and the prepared mixture as follows:

ee=([α−obs]/[α−max])×100.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

As used herein, unless otherwise noted, the term “salt” shall include any compound comprising related numbers of cations and anions, so that the resulting product is electrically neutral. The salt may be organic or inorganic, and may further be monatomic or polyatomic. Wherein the compound contains a basic moiety, the salt is an acid addition salt, which may, for example, be formed by mixing a solution of the compound containing the basic moiety (e.g. a quaternary nitrogen) with a solution of a suitably selected acid such as HCl, HBr, HI, TFA, H₂SO₄, and the like. Similarly, wherein the compound contains an acidic moiety, the salt is a base addition salt, which may, for example, be formed by mixing a solution of the compound containing the acidic moiety with a solution of a suitably selected base, such as an alkaline earth metal or quaternary ammonium ligand.

In certain embodiments, the present invention is directed to a salt of a compound of formula (I), a compound of formula (II), or the compound of formula (III), wherein the salt is an organic or inorganic acid addition salt such as a hydrochloride, hydrobromide, hydroiodide, trifluoroacetate, and the like. Preferably, the acid addition salt is a trifluoroacetate. In certain embodiments of the present invention, the salt is a pharmaceutically acceptable salt.

For use in medicine, the salts of the compounds of the present invention are non-toxic “pharmaceutically acceptable salts”. Other salts may, however, be useful in the preparation of compounds according to this invention or of their pharmaceutically acceptable salts.

Suitable pharmaceutically acceptable salts of the compounds of the present invention include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, trifluoroacetic acid, benzoic acid, citric acid, tartaric acid, carbonic acid or phosphoric acid. Furthermore, where the compounds of the present invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include, but are not limited to, the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide, tifluoroacetate and valerate.

Representative acids which may be used in the preparation of pharmaceutically acceptable salts include, but are not limited to, the following: acids including acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hipuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinc acid, nitric acid, oleic acid, orotic acid, oxalic acid, palmitic acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid.

Representative bases which may be used in the preparation of pharmaceutically acceptable salts include, but are not limited to, the following: bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

### General Synthesis Schemes

Compounds of formula (I) and compounds of formula (II) wherein A is

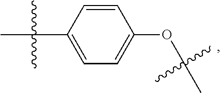 [SMILES: *Oc1ccc(*)cc1]

and the compound of formula (III), may be prepared from a common intermediate, the compound of formula (V)

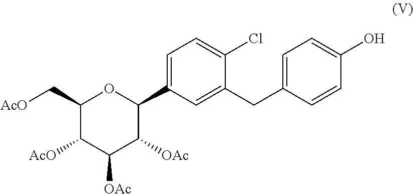 [SMILES: CC(=O)OC[C@H]1O[C@@H](c2ccc(Cl)c(Cc3ccc(O)cc3)c2)[C@H](OC(C)=O)[C@@H](OC(C)=O)[C@@H]1OC(C)=O]

also known as (2R,3R,4R,5S,6S)-2-(acetoxymethyl)-6-(4-chloro-3-(4-hydroxybenzyl)phenyl)tetrahydro-2H-pyran-3,4,5-triyl triacetate.

The compound of formula (V) may be prepared as described in Scheme 1, below.

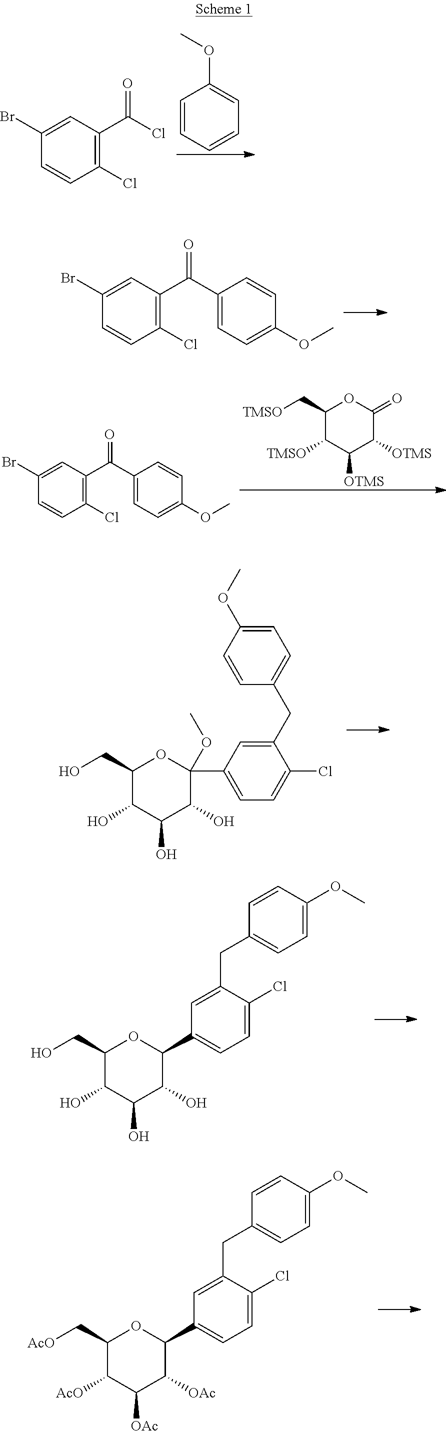

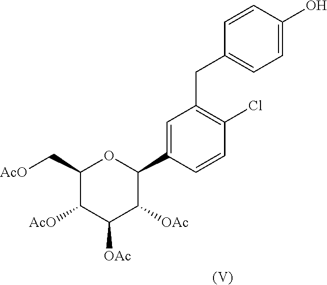 [SMILES: CC(=O)OC[C@H]1O[C@@H](c2ccc(Cl)c(Cc3ccc(O)cc3)c2)[C@H](OC(C)=O)[C@@H](OC(C)=O)[C@@H]1OC(C)=O ; "(V)"]

Accordingly, 5-bromo-2-chlorobenzoyl chloride, a known compound or compound prepared by known methods is reacted with anisole, a known compound, in the presence of a suitably selected Lewis acid such as AlCl₃, Al(OTf)₃, SnCl₄, and the like; in a suitably selected organic solvent such as DCM, ClCH₂CH₂Cl, CH₃NO₂, and the like; to yield (5-bromo-2-chlorophenyl)(4-methoxyphenyl)methanone.

The (5-bromo-2-chlorophenyl)(4-methoxyphenyl)methanone is reacted with a suitably selected reducing agent such as a mixture of TES (triethylsilane) and BF₃.Et₂O, a mixture of NaBH₄ and TFA, a mixture of N₂H₄ and NaOH, and the like; in a suitably selected organic solvent such as a mixture of DCM/acetonitrile, Et₂O, (CH₂OH)₂, and the like; to yield 4-bromo-1-chloro-2-(4-methoxybenzyl)benzene.

The 4-bromo-1-chloro-2-(4-methoxybenzyl)benzene is reacted with a suitably selected lithiating agent such as n-BuLi, and the like; in a suitably selected anhydrous organic solvent such as THF, Et₂O, 2-methyl-THF, and the like; and then reacted with (3R,4S,5R,6R)-3,4,5-tris((trimethylsilyl)oxy)-6-(((trimethylsilyl)oxy)methyl)tetrahydro-2H-pyran-2-one, a known compound or compound prepared by known methods; and then reacted with a suitably selected acid such as CH₃SO₃H, TfOH, and the like; to yield (3R,4S,5S,6R)-2-(4-chloro-3-(4-methoxybenzyl)phenyl)-6-(hydroxymethyl)-2-methoxytetrahydro-2H-pyran-3,4,5-triol.

The (3R,4S,5S,6R)-2-(4-chloro-3-(4-methoxybenzyl)phenyl)-6-(hydroxymethyl)-2-methoxytetrahydro-2H-pyran-3,4,5-triol is reacted with a suitably selected reducing agent such as a mixture of TES (triethylsilane) and BF₃.Et₂O, TFA, and the like; in a suitably selected organic solvent such as a mixture of DCM/acetonitrile, ClCH₂CH₂Cl, and the like; to yield (2S,3R,4R,5S,6R)-2-(4-chloro-3-(4-methoxybenzyl)phenyl)-6-(hydroxymethyl)tetrahydro-2H-pyran-3,4,5-triol.

The (2S,3R,4R,5S,6R)-2-(4-chloro-3-(4-methoxybenzyl)phenyl)-6-(hydroxymethyl)tetrahydro-2H-pyran-3,4,5-triol is reacted to install protecting groups on hydroxy groups of the glucopyranosyl, reacting with a suitably selected protecting reagent such as acetic anhydride (to protect with acetyl groups), AcCl, benzyl chloride, and the like; in the presence of a suitably selected nucleophilic catalyst such as DMAP, and the like; in a suitably selected organic solvent such as pyridine, Et₃N, DIEA, and the like; to yield (2R,3R,4R,5S,6S)-2-(acetoxymethyl)-6-(4-chloro-3-(4-methoxybenzyl)phenyl)tetrahydro-2H-pyran-3,4,5-triyl triacetate.

The (2R,3R,4R,5S,6S)-2-(acetoxymethyl)-6-(4-chloro-3-(4-methoxybenzyl)phenyl)tetrahydro-2H-pyran-3,4,5-triyl triacetate is reacted with a suitably selected demethylating agent such as BBr₃, AlCl₃, a mixture of BF₃.Et₂O/NaI, and the like; in a suitably selected organic solvent such as DCM, acetonitrile, and the like; to yield (2R,3R,4R,5S,6S)-2-(acetoxymethyl)-6-(4-chloro-3-(4-hydroxybenzyl)phenyl)tetrahydro-2H-pyran-3,4,5-triyl triacetate, the compound of formula (V).

Compounds of formula (I) wherein R¹ is selected from the group consisting of —(CH₂)_{b}—R², —CH₂CH₂—O—CH₂CH₂—R²,

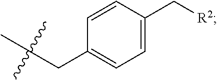

and wherein R² is selected from the group consisting of —N(CH₃)₃, 1-methyl-azetidin-1-yl, 1-methyl-pyrrolidin-1-yl, 1-methyl-piperidin-1-yl and 1-methyl-piperazin-1-yl; may be prepared as described in Scheme 2, below.

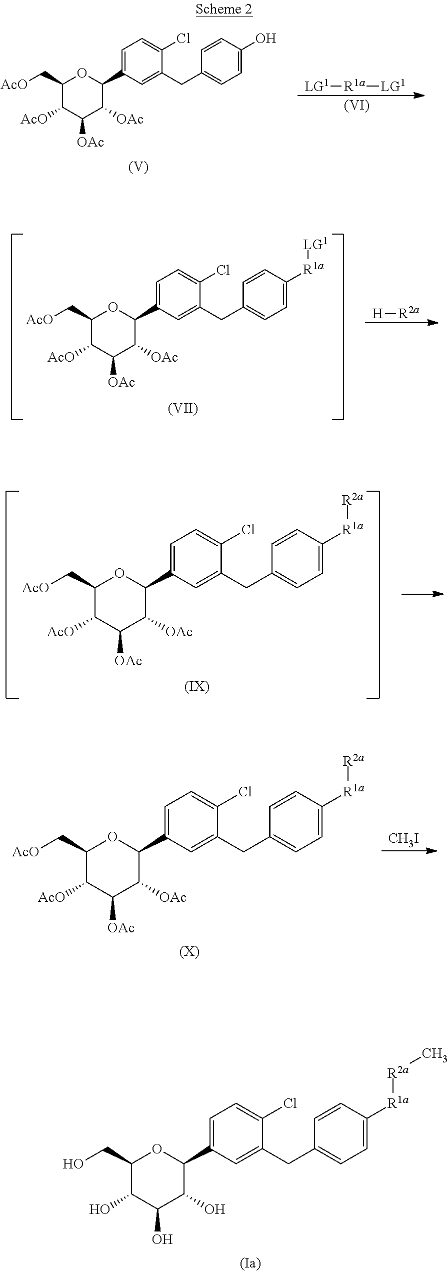

Accordingly, the compound of formula (V) is reacted with a suitably substituted compound of formula (VI), wherein R^{1a} is selected from the group consisting of —(CH₂)_{b}—, —CH₂CH₂—O—CH₂CH₂— and

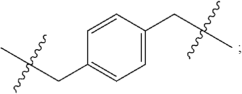 [SMILES: *Cc1ccc(C*)cc1]

and wherein both LG¹ are the same and are a suitably selected leaving groups such as Br, Cl, mesylate, tosylate, and the like, a known compound or compound prepared by known methods; wherein the compound of formula (VI) is present in an amount of at least 1 molar equivalent (relative to the moles of the compound of formula (V));

in the presence of a suitably selected base such as Ca₂CO₃, K₂CO₃, NaH, and the like; in a suitably selected organic solvent such as DMF, acetonitrile, THF, and the like; to yield the corresponding compound of formula (VII).

The compound of formula (VII) is reacted with a suitably substituted compound of formula (VIII), wherein R^{2a} is selected from the group consisting of —N(CH₃)₂, azetidin-1-yl, pyrrolidin-1-yl, piperidin-1-yl and piperazin-1-yl; a known compound or compound prepared by known methods; to yield the corresponding compound of formula (IX).

The compound of formula (IX) is de-protected according to known methods; to yield the corresponding compound of formula (X). For example, the compound of formula (IX) may be reacted with a suitably selected reagent such as sodium methoxide, in a suitably selected solvent such as methanol; to yield the corresponding compound of formula (X).

The compound of formula (X) is reacted with a suitably selected methylating agent such as CH₃I, Me₂SO₄ and the like; in the presence of a suitably selected base such as NaHCO₃, Cs₂CO₃, K₂CO₃, and the like; in a suitably selected solvent such as methanol, ethanol, acetone, and the like; to yield the corresponding compound of formula (Ia) (i.e. a compound of formula (I) wherein R¹ is selected from the group consisting of —(CH₂)_{b}—R², —CH₂CH₂—O—CH₂CH₂—R²,

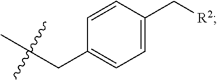

and wherein R² is selected from the group consisting of —N(CH₃)₃, 1-methyl-azetidin-1-yl, 1-methyl-pyrrolidin-1-yl, 1-methyl-piperidin-1-yl and 1-methyl-piperazin-1-yl).

One skilled in the art will recognize that the compounds of formula (I) prepared as described in Scheme 2 above contain a quaternary nitrogen atom, with a positive charge on said nitrogen atom, and are preferably isolated as their corresponding acid addition salt.

Compounds of formula (I) wherein R¹ is —CH₂CH₂CH₂—SO₃H may be prepared as described in Scheme 3, below.

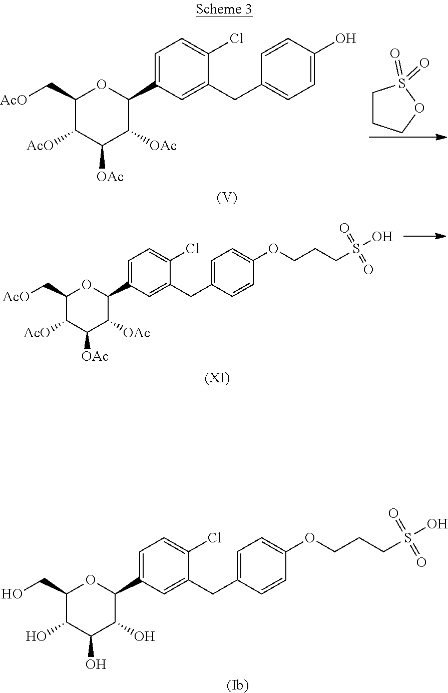

Accordingly, the compound of formula (V), prepared as described in Scheme 1 above, is reacted with 1,2-oxathiolane 2,2-dioxide; in the presence of a suitably selected base such as Cs₂CO₃, K₂CO₃, NaH, and the like; in a suitably selected organic solvent such as acetonitrile, DMF, THF, and the like; to yield the corresponding compound of formula (XI).

The compound of formula (XI) is de-protected according to known methods; to yield the corresponding compound of formula (Ib). For example, the compound of formula (XI) may be reacted with a suitably selected reagent such as sodium methoxide, in a suitably selected solvent such as methanol; to yield the corresponding compound of formula (Ib).

Compounds of formula (I) wherein R¹ is —CH₂CH₂—(OCH₂CH₂)ₐ—OCH₃ may be similarly prepared as described in Scheme 4 below.

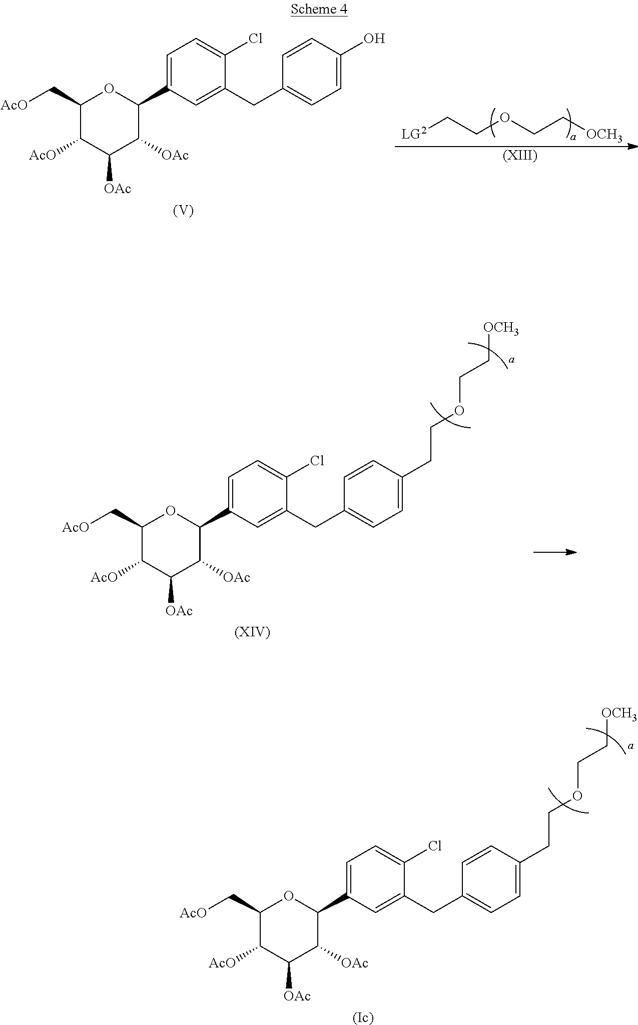

Accordingly, the compound of formula (V) is reacted with a suitably substituted compound of formula (XIII), wherein LG² is a suitably selected leaving group such as mesylate, tosylate, and the like; in the presence of a suitably selected base such as CsCO₃, K₂CO₃, NaH and the like; to yield the corresponding compound of formula (XIV).

The compound of formula (XIV) is de-protected according to known methods; to yield the corresponding compound of formula (Ic). For example, the compound of formula (XIV) may be reacted with a suitably selected reagent such as sodium methoxide, in a suitably selected solvent such as methanol; to yield the corresponding compound of formula (Ic).

Compound #3, a compound of formula (I) wherein R¹ is

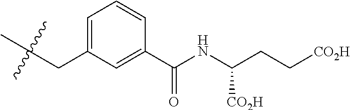 [SMILES: *Cc1cccc(C(=O)N[C@H](CCC(=O)O)C(=O)O)c1]

may be prepared as described in Example 4, which follows hereinafter, or as described in Scheme 5, below.

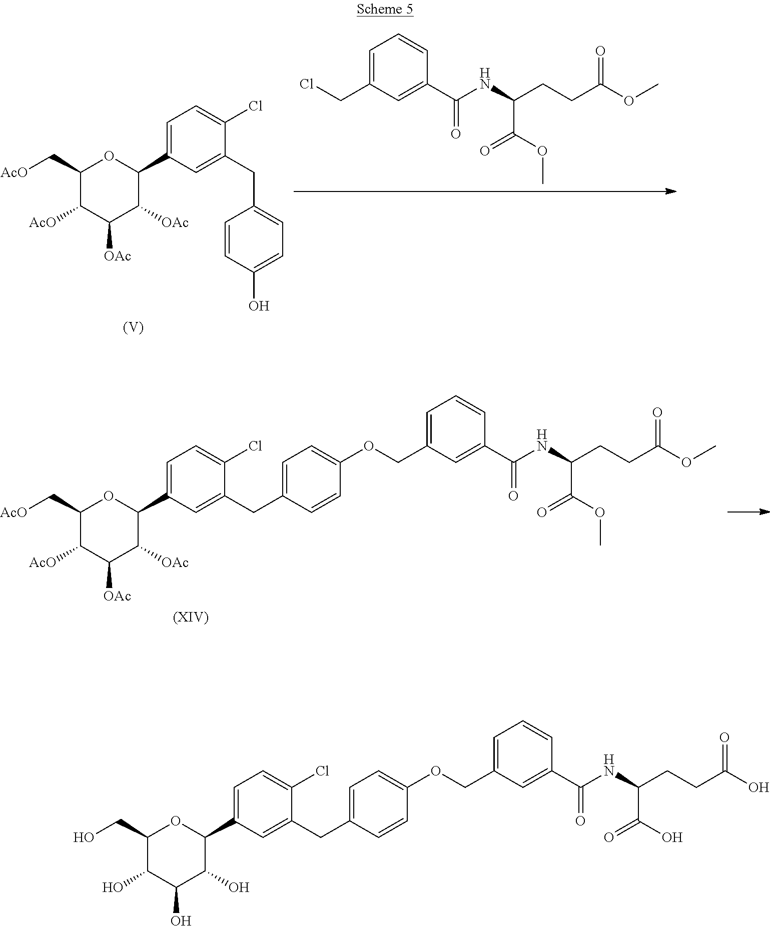

Accordingly, the compound of formula (V) is reacted with dimethyl (3-(chloromethyl)benzoyl)-L-glutamate, a known compound; in the presence of a suitably selected base such as Cs₂CO₃, K₂CO₃, NaH, and the like; in a suitably selected organic solvent such as DMF, acetonitrile, THF, and the like; to yield dimethyl (3-((4-(2-chloro-5-((2S,3S,4R,5R,6R)-3,4,5-triacetoxy-6-(acetoxymethyl)tetrahydro-2H-pyran-2-yl)benzyl)phenoxy)methyl)benzoyl)-L-glutamate.

The dimethyl (3-((4-(2-chloro-5-((2S,3S,4R,5R,6R)-3,4,5-triacetoxy-6-(acetoxymethyl)tetrahydro-2H-pyran-2-yl)benzyl)phenoxy)methyl)benzoyl)-L-glutamate is reacted with a suitably selected base such as sodium methoxide, sodium ethoxide, NaOH, and the like; in a suitably selected organic solvent such as MeOH, water, and the like; to yield (3-((4-(2-chloro-5-((2S,3R,4R,5S,6R)-3,4,5-trihydroxy-6-(hydroxymethyl)tetrahydro-2H-pyran-2-yl)benzyl)phenoxy)methyl)benzoyl)-L-glutamic acid.

Compounds of formula (II) wherein A is

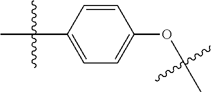 [SMILES: *Oc1ccc(*)cc1]

and wherein L¹ is selected from the group consisting of —CH₂CH₂—(OCH₂CH₂)_{c}—, —CH(CO₂H)—CH₂CH₂—CH(CO₂H)—,

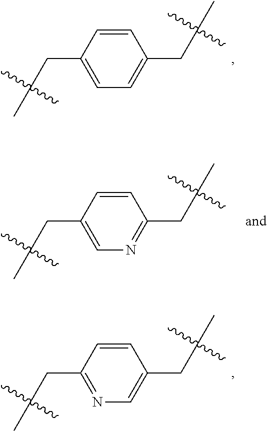 [SMILES: *Cc1ccc(C*)cc1 ; *Cc1ccc(C*)nc1 ; *Cc1ccc(C*)nc1]

may be prepared according to the procedure described in Scheme 6, below.

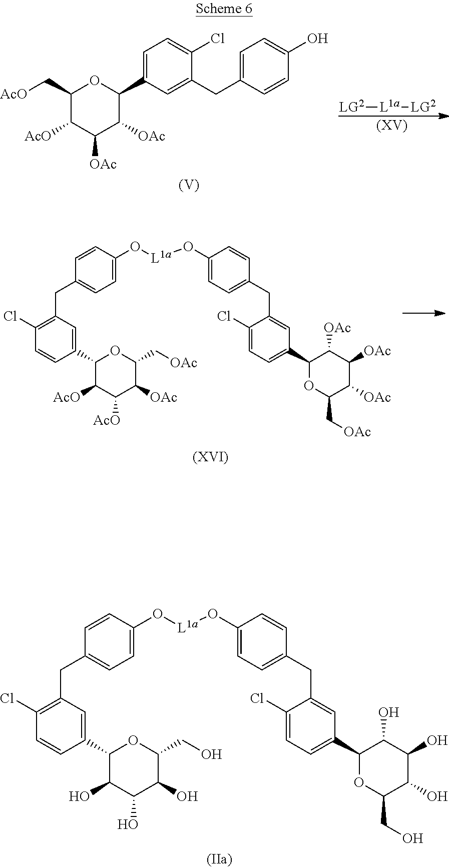

Accordingly, the compound of formula (V) is reacted with a suitably substituted compound of formula (XV), wherein L^{1a} is selected from the group consisting of —CH₂CH₂—(OCH₂CH₂)_{c}—, —CH(CO₂H)—CH₂CH₂—CH(CO₂H)—,

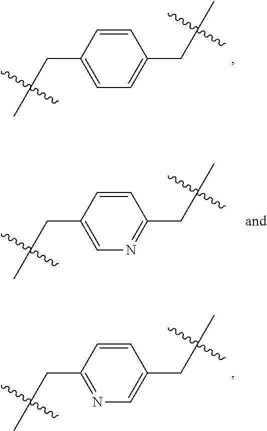 [SMILES: *Cc1ccc(C*)cc1 ; *Cc1ccc(C*)nc1 ; *Cc1ccc(C*)nc1]

and wherein each LG² is the same and is a suitably selected leaving group such as Br, Cl, mesylate, tosylate, and the like, a known compound or compound prepared by known methods; wherein the compound of formula (XV) is present in an amount of about 0.5 molar equivalents (relative to the amount of the compound of formula (V);

in the presence of a suitably selected base such as Cs₂CO₃, K₂CO₃, NaH, and the like; in a suitably selected organic solvent such as DMF, acetonitrile, THF, and the like; to yield the corresponding compound of formula (XVI).

The compound of formula (XVI) is de-protected according to known methods; to yield the corresponding compound of formula (IIa) (i.e. a compound of formula (II) wherein L¹ is selected from the group consisting of —CH₂CH₂—(OCH₂CH₂)_{c}—, —CH(CO₂H)—CH₂CH₂—CH(CO₂H)—,

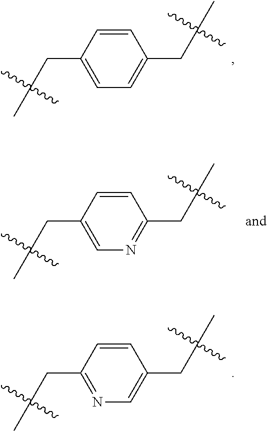 [SMILES: *Cc1ccc(C*)cc1 ; *Cc1ccc(C*)nc1 ; *Cc1ccc(C*)nc1]

For example, the compound of formula (XVI) may be reacted with a suitably selected reagent such as sodium methoxide, in a suitably selected solvent such as methanol; to yield the corresponding compound of formula (IIa).

Compounds of formula (II) wherein A is

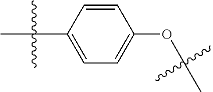 [SMILES: *Oc1ccc(*)cc1]

and wherein L¹ is selected from the group consisting of —CH₂CH₂—N⁺(CH₃)₂—CH₂CH₂—,

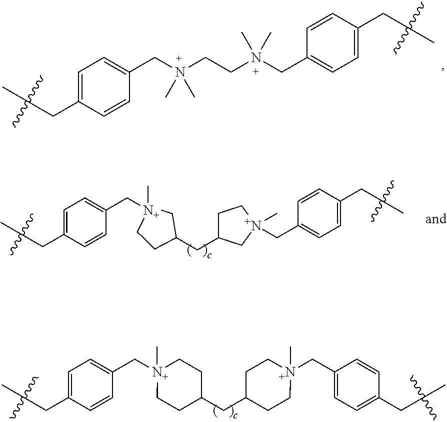

may be prepared as described in Scheme 7, below.

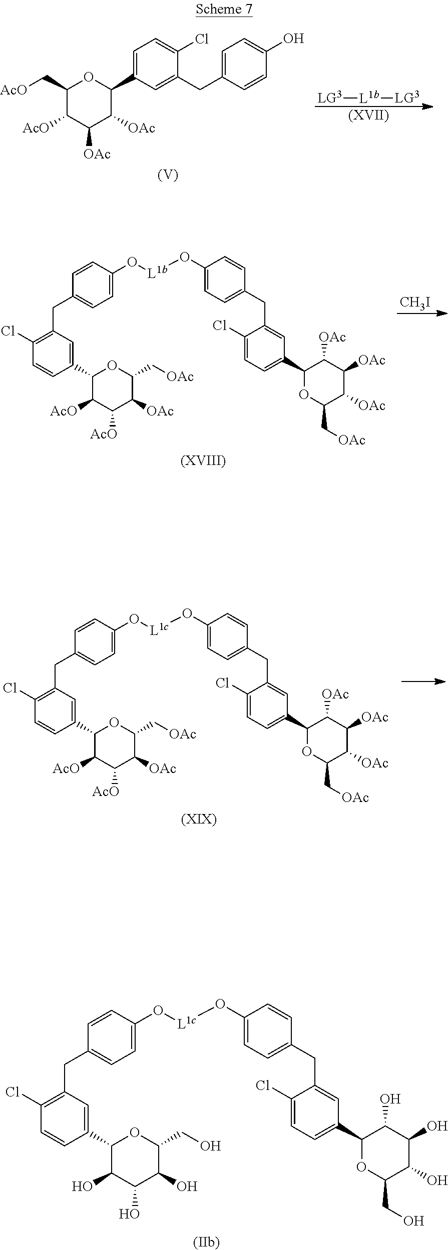

Accordingly, the compound of formula (V) is reacted with a suitably substituted compound of formula (XVII) wherein L^{1b} is selected from the group consisting of —CH₂CH₂—N(CH₃)—CH₂CH₂—,

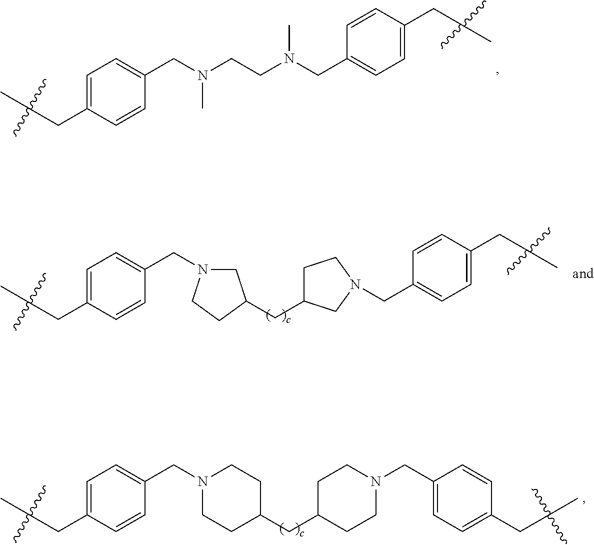

and wherein each LG³ is the same and is a suitably selected leaving group such as Br, Cl, mesylate, tosylate, and the like, a known compound or compound prepared by known methods; wherein the compound of formula (XV) is present in an amount of about 0.5 molar equivalents (relative to the amount of the compound of formula (V);

in the presence of a suitably selected coupling agent such as Cs₂CO₃, K₂CO₃, NaH, and the like; in a suitably selected organic solvent such as DMF, acetonitrile, THF, and the like; to yield the corresponding compound of formula (XVIII).

The compound of formula (XVIII) is reacted with a suitably selected methylating agent such as CH₃I, and the like; wherein the methylating agent is preferably present in a molar amount equal to greater than the number of nitrogen atom in the structure L^{1b} (for example, when L^{1b} is —CH₂CH₂—N(CH₃)—CH₂CH₂—, then the methylating agent is preferably present in amount equal to or greater than about one molar equivalent, whereas when L^{2b} is

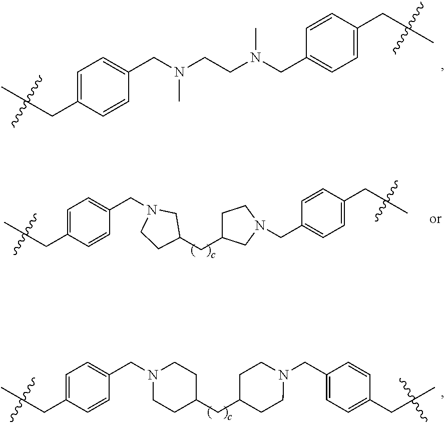

then the methylating agent is preferably present in amount equal to or greater than about two molar equivalents)

in the presence of a suitably selected base such as NaHCO₃, K₂CO₃, pyridine, and the like; in a suitably selected solvent such as methanol, ethanol, acetone, and the like; to yield the corresponding compound of formula (XIX), wherein L^{1c} represents the corresponding L^{1b} group wherein each nitrogen atom on L^{1b} group has been methylated (i.e. substituted with a methyl to produce a quaternary nitrogen).

The compound of formula (XIX) is de-protected according to known methods; to yield the corresponding compound of formula (IIb). For example, the compound of formula (XIX) may be reacted with a suitably selected reagent such as sodium methoxide, in a suitably selected solvent such as methanol; to yield the corresponding compound of formula (IIb) (i.e. the compound of formula (II) wherein L¹ is selected from the group consisting of —CH₂CH₂—N⁺(CH₃)₂—CH₂CH₂—,

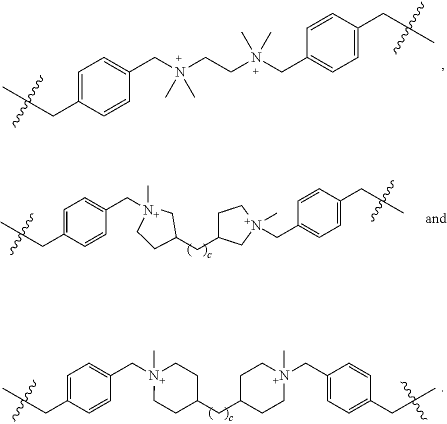

One skilled in the art will recognize that the compounds of formula (I) prepared as described in Scheme 7 above contain two quaternary nitrogen atoms, with a positive charge on each of said nitrogen atoms, and are preferably isolated as their corresponding acid addition salt.

Compounds of formula (II) wherein A is

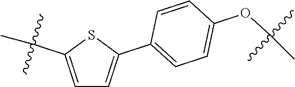 [SMILES: *Oc1ccc(-c2ccc(*)s2)cc1]

may be similarly prepared according to the procedures described in Schemes 6-7 above, substituting a compound of formula (X)

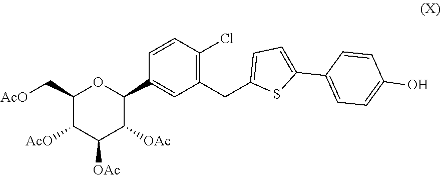 [SMILES: CC(=O)OC[C@H]1O[C@@H](c2ccc(Cl)c(Cc3ccc(-c4ccc(O)cc4)s3)c2)[C@H](OC(C)=O)[C@@H](OC(C)=O)[C@@H]1OC(C)=O]

also known as (2R,3R,4R,5S,6S)-2-(acetoxymethyl)-6-(4-chloro-3-((5-(4-hydroxyphenyl)thiophen-2-yl)methyl)phenyl)tetrahydro-2H-pyran-3,4,5-triyl triacetate, for the compound of formula (V), and reacting as described.

The compound of formula (X) may be prepared as described in Scheme 8, below.

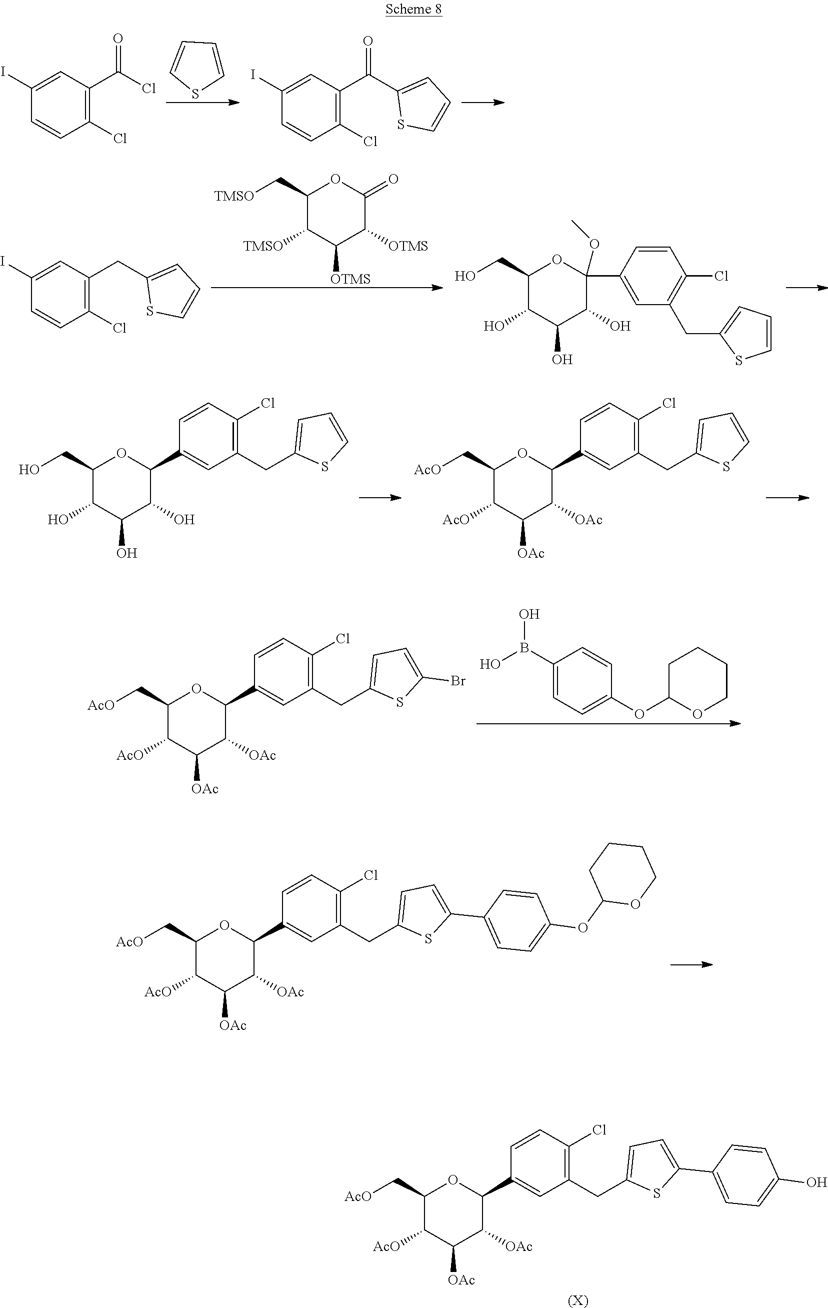

Accordingly, 2-chloro-5-iodobenzoyl chloride, a known compound or compound prepared by known methods, is reacted with thiophene, a known compound; in the presence of a suitably selected Lewis acid such as AlCl₃, Al(OTf)₃, FeCl₃, SnCl₄, TFAA/H₃PO₄, and the like; in a suitably selected organic solvent such as DCM, ClCH₂CH₂Cl, CH₃NO₂, and the like; to yield (2-chloro-5-iodophenyl)(thiophen-2-yl)methanone.

The (2-chloro-5-iodophenyl)(thiophen-2-yl)methanone is reacted with a suitably selected reducing agent such as a mixture of TES (triethylsilane) and BF₃.Et₂O, a mixture of NaBH₄ and TFA, a mixture of N₂H₄ and NaOH, and the like; in a suitably selected organic solvent such as a mixture of DCM/acetonitrile, Et₂O, (CH₂OH)₂, and the like; to yield 2-(2-chloro-5-iodobenzyl)thiophene.

The 2-(2-chloro-5-iodobenzyl)thiophene is reacted with a suitably selected metal-exchange reagent such as i-PrMgCl.LiCl, n-BuLi, and the like; in a suitably selected organic solvent such as THF, Et₂O, 2-methyl-THF, and the like; and then reacted with (3R,4S,5R,6R)-3,4,5-tris((trimethylsilyl)oxy)-6-(((trimethylsilyl)oxy)methyl)tetrahydro-2H-pyran-2-one, a known compound or compound prepared by known methods; and then reacted with a suitably selected acid such as CH₃SO₃H, TfOH, and the like; to yield (3R,4S,5S,6R)-2-(4-chloro-3-(thiophen-2-ylmethyl)phenyl)-6-(hydroxymethyl)-2-methoxytetrahydro-2H-pyran-3,4,5-triol.

The (3R,4S,5S,6R)-2-(4-chloro-3-(thiophen-2-ylmethyl)phenyl)-6-(hydroxymethyl)-2-methoxytetrahydro-2H-pyran-3,4,5-triol is reacted with a suitably selected reducing agent such as a mixture of TES (triethylsilane) and BF₃.Et₂O, TFA, and the like; in a suitably selected organic solvent or mixture of organic solvents such as DCM/acetonitrile, ClCH₂CH₂Cl, and the like; to yield (2S,3R,4R,5S,6R)-2-(4-chloro-3-(thiophen-2-ylmethyl)phenyl)-6-(hydroxymethyl)tetrahydro-2H-pyran-3,4,5-triol.

The (2S,3R,4R,5S,6R)-2-(4-chloro-3-(thiophen-2-ylmethyl)phenyl)-6-(hydroxymethyl)tetrahydro-2H-pyran-3,4,5-triol is reacted to install protecting groups on hydroxy groups of the glucopyranosyl, reacting with a suitably selected protecting reagent such as acetic anhydride (to protect with acetyl groups), AcCl, benzyl chloride, and the like; in the presence of a suitably selected nucleophilic catalyst such as DMAP, and the like; in a suitably selected organic solvent such as pyridine, Et₃N, DIEA, and the like; to yield (2R,3R,4R,5S,6S)-2-(acetoxymethyl)-6-(4-chloro-3-(thiophen-2-ylmethyl)phenyl)tetrahydro-2H-pyran-3,4,5-triyltriacetate.

The (2R,3R,4R,5S,6S)-2-(acetoxymethyl)-6-(4-chloro-3-(thiophen-2-ylmethyl)phenyl)tetrahydro-2H-pyran-3,4,5-triyl triacetate is reacted with a suitably selected brominating agent such as NBS, PyHBr₃, Br₂, and the like; in a suitably selected organic solvent such as acetonitrile, DMF, CH₂Cl₂, and the like; to yield (2R,3R,4R,5S,6S)-2-(acetoxymethyl)-6-(3-((5-bromothiophen-2-yl)methyl)-4-chlorophenyl)tetrahydro-2H-pyran-3,4,5-triyltriacetate.

The (2R,3R,4R,5S,6S)-2-(acetoxymethyl)-6-(3-((5-bromothiophen-2-yl)methyl)-4-chlorophenyl)tetrahydro-2H-pyran-3,4,5-triyltriacetate is reacted with (4-((tetrahydro-2H-pyran-2-yl)oxy)phenyl)boronic acid, a known compound or compound prepared by known methods; in the presence of a suitably selected coupling agent such as PdCl₂(dppf)₂, Pd(PPh₃)₄, Pd₂dba₃, and the like; in the presence of a suitably selected base such as K₂CO₃, CsF, K₃PO₄, and the like; in a suitably selected organic solvent such as toluene, 1,4-dioxane, DMF, and the like; to yield (2R,3R,4R,5S,6S)-2-(acetoxymethyl)-6-(4-chloro-34(5-(4-((tetrahydro-2H-pyran-2-yl)oxy)phenyl)thiophen-2-yl)methyl)phenyl)tetrahydro-2H-pyran-3,4,5-triyltriacetate.

The (2R,3R,4R,5S,6S)-2-(acetoxymethyl)-6-(4-chloro-3-((5-(4-((tetrahydro-2H-pyran-2-yl)oxy)phenyl)thiophen-2-yl)methyl)phenyl)tetrahydro-2H-pyran-3,4,5-triyltriacetate is reacted with a suitably selected acid such as HCl in THF, TFA, TsOH, and the like; in a suitably selected organic solvent such as methanol, THF, acetonitrile, and the like; to yield (2R,3R,4R,5S,6S)-2-(acetoxymethyl)-6-(4-chloro-34(5-(4-hydroxyphenyl)thiophen-2-yl)methyl)phenyl)tetrahydro-2H-pyran-3,4,5-triyltriacetate, the compound of formula (X).

The compound of formula (III) may be prepared as described in Example 7, which follows hereinafter.

### Pharmaceutical Compositions

The present invention further comprises pharmaceutical compositions containing one or more compounds of formula (I), formula (II) or formula (III) with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus, for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.01 mg to about 1000 mg or any amount or range therein, and may be given at a dosage of from about 0.05 mg/day to about 500 mg/day, or any amount or range therein, preferably from about 0.1 mg/day to about 100 mg/day, or any amount or range therein, preferably from about 1 mg/day to about 50 mg/day, or any amount or range therein. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid pre-formulation composition is then subdivided into unit dosage forms of the type described above containing from about 0.01 mg to about 1,000 mg, or any amount or range therein, of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavored syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of treating disorders mediated by SGLT2 activity, described in the present invention may also be carried out using a pharmaceutical composition comprising any of the compounds as defined herein and a pharmaceutically acceptable carrier. The pharmaceutical composition may contain between about 0.01 mg and about 1000 mg of the compound, or any amount or range therein, preferably from about 0.05 mg to about 500 mg of the compound, or any amount or range therein, more preferably from about 0.1 mg to about 100 mg of the compound, or any amount or range therein, more preferably from about 0.1 mg to about 50 mg of the compound, or any amount or range therein; and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

To prepare a pharmaceutical composition of the present invention, a compound of formula (I), formula (II) or formula (III) as the active ingredient is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

Compounds of this invention may be administered in any of the foregoing compositions and according to dosage regimens established in the art whenever treatment of disorders mediated by SGLT2 activity, is required.

The daily dosage of the products may be varied over a wide range from about 0.01 mg to about 1,000 mg per adult human per day, or any amount or range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the patient to be treated. An effective amount of the drug may be ordinarily supplied at a dosage level of from about 0.005 mg/kg to about 10 mg/kg of body weight per day, or any amount or range therein. Preferably, the range is from about 0.01 to about 5.0 mg/kg of body weight per day, or any amount or range therein, more preferably, from about 0.1 to about 1.0 mg/kg of body weight per day, or any amount or range therein, more preferably, from about 0.1 to about 0.5 mg/kg of body weight per day, or any amount or range therein. The compounds may be administered on a regimen of 1 to 4 times per day.

Optimal dosages to be administered may be readily determined by those skilled in the art, and will vary with the particular compound used, the mode of administration, the strength of the preparation, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

The following Examples are set forth to aid in the understanding of the invention, and are not intended and should not be construed to limit in any way the invention set forth in the claims which follow thereafter.

In the Examples which follow, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like.

### EXAMPLE 1

### Compound #10

### (2R,2′R,3S,3′S,4R,4′R,5R,5′R,6S,6′S)-6,6′-(((((oxybis(ethane-2,1-diyl))bis(oxy))bis(4,1-phenylene))bis(methylene))bis(4-chloro-3,1-phenylene))bis(2-(hydroxymethyl)tetrahydro-2H-pyran-3,4,5-triol)

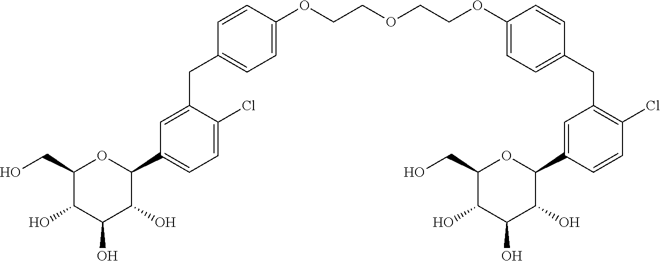 [SMILES: OC[C@H]1O[C@@H](c2ccc(Cl)c(Cc3ccc(OCCOCCOc4ccc(Cc5cc([C@@H]6O[C@H](CO)[C@@H](O)[C@H](O)[C@H]6O)ccc5Cl)cc4)cc3)c2)[C@H](O)[C@@H](O)[C@@H]1O]

### Step 1: (5-Bromo-2-chlorophenyl)(4-methoxyphenyl)methanone

To a suspension of 5-bromo-2-chlorobenzoic acid (6.24 g, 26.5 mmol) in DCM (50 mL) was added oxalyl chloride (2.9 mL, 31.8 mmol) and the mixture was stirred at room temperature for 5 min. Then 3 drops of DMF (˜0.1 ml) was added and the resulting mixture was stirred at room temperature for 2 h. The volatile was removed under reduced pressure to yield 5-bromo-2-chlorobenzoyl chloride as a colorless solid, which was dissolved in DCM (50 mL). To this solution were then added anisole (3.025 ml, 27.83 mmol) and then AlCl₃ (3.61 g, 27.04 mol) portionwise, so that the temperature did not exceed −4° C. The mixture was then stirred at 5° C. for 3 h, then poured into ice water and extracted with CHCl₃ three times. The combined organic layers were washed with 1M hydrochloric acid, water and brine, then dried over MgSO₄ and concentrated. The residual solid was crystallized from a 4:1 mixture of hexane and ethyl acetate to yield (5-bromo-2-chlorophenyl)(4-methoxyphenyl)methanone as a white solid. ¹H NMR (400 MHz, CHLOROFORM-d) δ 7.78 (d, J=8.59 Hz, 2H), 7.52-7.57 (m, 1H), 7.49 (s, 1H), 7.34 (s, 1H), 7.26 (s, 1H), 6.95 (d, J=8.59 Hz, 2H), 3.89 (s, 3H). m/z (MH⁺): 326.85.

### Step 2: 4-Bromo-1-chloro-2-(4-methoxybenzyl)benzene

(5-Bromo-2-chlorophenyl)(4-methoxyphenyl)methanone (7.61 g, 23.4 mmol) was dissolved in DCM/Acetonitrile (1:1 v/v, 60 mL). To the mixture was then add triethylsilane (9.71 mL, 60.77 mmol), followed by BF₃.Et₂O dropwise and the resulting pink mixture was stirred at room temperature for 40 min, then stirred at 45° C. overnight. The reaction was quenched with aqueous NaHCO₃, the organic layer was separated and the aqueous layer extracted with EtOAc three times. The combined extracts were dried with Na₂SO₄ and the resulting solid was filtered, then concentrated. The residue was purified by flash column chromatography on silica gel (120 g, EtOAc/heptane: 0>>>5%) to yield 4-bromo-1-chloro-2-(4-methoxybenzyl)benzene as a colorless oil. ¹H NMR (400 MHz, CHLOROFORM-d) δ 7.20-7.30 (m, 3H), 7.10 (d, J=8.59 Hz, 2H), 6.82-6.89 (m, 2H), 3.99 (s, 2H), 3.79 (s, 3H).

### Step 3: (3R,4S,5S,6R)-2-(4-chloro-3-(4-methoxybenzyl)phenyl)-6-(hydroxymethyl)-2-methoxytetrahydro-2H-pyran-3,4,5-triol

A solution of 4-bromo-1-chloro-2-(4-methoxybenzyl)benzene (2.42 g, 7.8 mmol) in anhydrous THF (6 mL) was cooled to −78° C. The reaction vessel was evacuated, re-filled with argon and this process was repeated twice. To the reaction mixture was then added n-BuLi (1.6 M in hexane, 4.85 ml) dropwise and the resulting mixture was stirred at −78° C. under argon for 30 min. A solution of (3R,4S,5R,6R)-3,4,5-tris((trimethylsilyl)oxy)-6-(((trimethylsilyl)oxy)methyl)tetrahydro-2H-pyran-2-one (3.62 g, 7.75 mmol) in THF (9 mL) was added dropwise and the resulting reaction mixture was maintained with stirring at −78° C. under argon for 1.5 h. To the mixture was then added 0.58 N CH₃SO₃H solution in MeOH (16 mL) at −78° C. and the resulting mixture was stirred at 78° C. for 5 min, then slowly warmed to room temperature for 48 h. The progress of the reaction was monitored by LC/MS. The reaction was then quenched with saturated aqueous NaHCO₃ solution (50 ml), extracted with EtOAc three times (50 ml) and the combined extracts were washed with brine, dried with Na₂SO₄, the insoluble solid was filtered off and the filtrate was concentrated under reduced pressure to yield a white foam, which was used for the next step reaction without further purification.

### Step 4: (2S,3R,4R,5S,6R)-2-(4-chloro-3-(4-methoxybenzyl)phenyl)-6-(hydroxymethyl)tetrahydro-2H-pyran-3,4,5-triol

(3R,4S,5S,6R)-2-(4-chloro-3-(4-methoxybenzyl)phenyl)-6-(hydroxymethyl)-2-methoxytetrahydro-2H-pyran-3,4,5-triol (3.29 g, 7.75 mmol) was dissolved in DCM/acetonitrile (v/v 1:1, 60 mL) and the resulting solution was cooled to 0° C. To the mixture was then added triethylsilane (2.72 ml, 17.06 mmol), followed by BF₃.Et₂O dropwise and the resulting mixture was stirred at 0° C. for 1 h. The reaction mixture was quenched with aqueous NaHCO₃, extracted with EtOAc three times and the combined extracts were dried with Na₂SO₄. The solid was filtered off and the filtrate was concentrated under reduced pressure. The residue was purified by flash column chromatography on silica gel (80 g, EtOAc/heptane: 0>>>100%, then 1% MeOH in EtOAc) to yield (2S,3R,4R,5S,6R)-2-(4-chloro-3-(4-methoxybenzyl)phenyl)-6-(hydroxymethyl)tetrahydro-2H-pyran-3,4,5-triol as a white solid.

### Step 5: (2R,3R,4R,5S,6S)-2-(acetoxymethyl)-6-(4-chloro-3-(4-methoxybenzyl)phenyl)tetrahydro-2H-pyran-3,4,5-triyl triacetate

To a solution of (2S,3R,4R,5S,6R)-2-(4-chloro-3-(4-methoxybenzyl)phenyl)-6-(hydroxymethyl)tetrahydro-2H-pyran-3,4,5-triol (1.66 g, 4.20 mmol) and pyridine (3.39 ml, 42.02 mmol) in DCM (40 ml) was added DMAP (25.7 mg, 0.21 mmol), followed by acetic anhydride (4 ml, 42.02 mmol) and the resulting mixture was stirred at room temperature for 16 h. Aqueous NaHCO₃ solution (20 ml) was added and the mixture was stirred at room temperature for 30 min. The organic layer was then separated and the aqueous layer was extracted with DCM three times. The combined DCM extracts were washed with 1N HCl three times, dried with Na₂SO₄ and the insoluble solid was filtered off. The filtrate was concentrated under reduced pressure to yield a white foam which was a mixture of two isomers/alpha-anomer and beta-anomer. The white foam (mixture) was dissolved in hot EtOH. A white solid was crystallized from EtOH while being cooled to −40° C. This solid was filtered off and dried in vacuo to yield (2R,3R,4R,5S,6S)-2-(acetoxymethyl)-6-(4-chloro-3-(4-methoxybenzyl)phenyl)tetrahydro-2H-pyran-3,4,5-triyl triacetate as a white solid. ¹H NMR (400 MHz, CHLOROFORM-d) δ 7.33-7.38 (m, 1H), 7.26 (s, 1H), 7.19 (dd, J=2.02, 8.08 Hz, 1H), 7.03-7.10 (m, 2H), 6.81-6.86 (m, 2H), 5.25-5.32 (m, 1H), 5.15-5.24 (m, 1H), 5.01-5.10 (m, 1H), 4.32 (d, J=9.60 Hz, 1H), 4.23-4.29 (m, 1H), 4.11-4.17 (m, 1H), 3.95-4.08 (m, 2H), 3.80-3.85 (m, 1H), 3.78 (s, 3H), 2.08 (s, 3H), 2.05 (s, 3H), 1.99 (s, 3H), 1.71 (s, 3H). m/z (M+Na): 585.0.

### Step 6: (2R,3R,4R,5S,6S)-2-(acetoxymethyl)-6-(4-chloro-3-(4-hydroxybenzyl)phenyl)tetrahydro-2H-pyran-3,4,5-triyl triacetate

(2R,3R,4R,5S,6S)-2-(acetoxymethyl)-6-(4-chloro-3-(4-methoxybenzyl)phenyl)tetrahydro-2H-pyran-3,4,5-triyl triacetate (1220 mg, 2.17 mmol) was dissolved in anhydrous DCM (20 mL), and the mixture cooled to −78° C. To the mixture was then added BBr₃ (13 mL, 13 mmol) at −78° C. and the mixture was stirred at −78° C. for 2 h, then at −30° C. for 1 h, and at 0° C. for 1 hour. The reaction mixture was diluted with aqueous NaHCO₃ solution, then extracted with EtOAc three times. The combined organic extracts were washed with brine, dried with Na₂SO₄. The solid was filtered off and the filtrate was concentrated and the residue was purified by flash column chromatography on silica gel (40 g, EtOAc/heptane: 0>>>55%>>>70%) to yield (2R,3R,4R,5S,6S)-2-(acetoxymethyl)-6-(4-chloro-3-(4-hydroxybenzyl)phenyl)tetrahydro-2H-pyran-3,4,5-triyl triacetate as a white foam. ¹H NMR (400 MHz, CHLOROFORM-d) δ 7.35 (d, J=8.08 Hz, 1H), 7.16-7.21 (m, 1H), 7.07 (d, J=2.02 Hz, 1H), 7.02 (d, J=8.59 Hz, 2H), 6.73-6.79 (m, 2H), 5.25-5.32 (m, 1H), 5.16-5.24 (m, 1H), 5.05 (t, J=9.60 Hz, 1H), 4.71 (s, 1H), 4.32 (d, J=10.11 Hz, 1H), 4.23-4.29 (m, 1H), 4.10-4.17 (m, 1H), 3.93-4.08 (m, 2H), 3.74-3.83 (m, 1H), 2.08 (s, 3H), 2.05 (s, 3H), 1.99 (s, 3H), 1.72 (s, 3H); m/z (M+Na): 571.05.

### Step 7: (2R,2′R,3S,3′S,4R,4′R,5R,5′R,6S,6′S)-6,6′-(((((oxybis(ethane-2,1-diyl))bis(oxy))bis(4,1-phenylene))bis(methylene))bis(4-chloro-3,1-phenylene))bis(2-(hydroxymethyl)tetrahydro-2H-pyran-3,4,5-triol)

A 10 ml vial was charged with (2R,3R,4R,5S,6S)-2-(acetoxymethyl)-6-(4-chloro-3-(4-hydroxybenzyl)phenyl)tetrahydro-2H-pyran-3,4,5-triyl triacetate (50 mg, 0.09 mmol), 1-bromo-2-(2-bromoethoxy)ethane and Cs₂CO₃ (74.2 mg, 0.228 mmol). To the mixture was then added anhydrous DMF (1 mL) and the vial was capped, and stirred at 50° C. for 1 h. The resulting mixture was then diluted with EtOAc, washed with 1N HCl and the organic layer was concentrated. The residue was purified by flash column chromatography on silica gel (12 g, EtOAc/heptane: 0>>>50%>>>100%) and the resulting white solid was suspended in MeOH. To the resulting mixture was then added added 25% NaOCH₃ in MeOH (3 drops) and the mixture was stirred at room temperature for 2 h. The solvent was removed under reduced pressure and the residue was purified by Gilson HPLC to yield (2R,2′R,3S,3′S,4R,4′R,5R,5′R,6S,6′S)-6,6′-(((((oxybis(ethane-2,1-diyl))bis(oxy))bis(4,1-phenylene))bis(methylene))bis(4-chloro-3,1-phenylene))bis(2-(hydroxymethyl)tetrahydro-2H-pyran-3,4,5-triol) as a white solid.

¹H NMR (400 MHz, METHANOL-d4) Shift 7.30-7.36 (m, 4H), 7.24-7.29 (m, 2H), 7.09 (d, J=8.59 Hz, 4H), 6.76-6.86 (m, 4H), 3.98-4.12 (m, 10H), 3.81-3.90 (m, 6H), 3.64-3.72 (m, 2H), 3.35-3.46 (m, 6H), 3.24-3.29 (m, 2H). m/z (MH+); 831.15.

### EXAMPLE 2

### Compound #5

### 1-(4-(4-(2-chloro-5-((2S,3R,4R,5S,6R)-3,4,5-trihydroxy-6-(hydroxymethyl)tetrahydro-2H-pyran-2-yl)benzyl)phenoxy)butyl)-1-methylpyrrolidin-1-ium trifluoroacetate

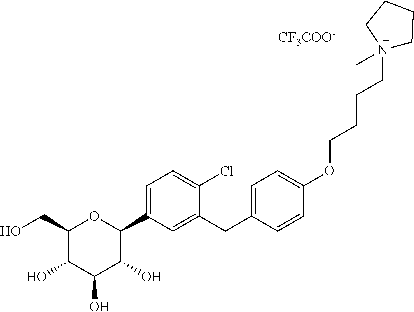

### Step 1: (2S,3R,4R,5S,6R)-2-(4-chloro-3-(4-(4-(pyrrolidin-1-yl)butoxy)benzyl)phenyl)-6-(hydroxymethyl)tetrahydro-2H-pyran-3,4,5-triol

A 10 ml vial was charged with (2R,3R,4R,5S,6S)-2-(acetoxymethyl)-6-(4-chloro-3-(4-hydroxybenzyl)phenyl)tetrahydro-2H-pyran-3,4,5-triyl triacetate (57.8 mg, 0.11 mmol), 1,4-dibromobutane (22.7 mg, 0.11 mmol) and Cs₂CO₃ (68.6 mg, 0.21 mmol). To the mixture was then added acetonitrile (1 mL) and the vial was capped, the mixture stirred at 70° C. for 1 h, then at room temperature for 16 h. Pyrrolidine (15 mg, 0.21 mmol) was added and the mixture was stirred at 70° C. for 30 mins. Upon completion of the reaction as indicated by LC/MS analysis, NaOMe solution (25% in MeOH, 0.05 mL) was added and the resulting mixture was stirred at 70° C. for 1 h. The resulting precipitate was filtered off and the filtrate was subjected to Gilson HPLC purification to yield a colorless syrup.

### Step 2: 1-(4-(4-(2-chloro-5-((2S,3R,4R,5S,6R)-3,4,5-trihydroxy-6-(hydroxymethyl)tetrahydro-2H-pyran-2-yl)benzyl)phenoxy)butyl)-1-methylpyrrolidin-1-ium trifluoroacetate

(2S,3R,4R,5S,6R)-2-(3-chloro-4-(4-(4-(pyrrolidin-1-yl)butoxy)benzyl)phenyl)-6-(hydroxymethyl)tetrahydro-2H-pyran-3,4,5-triol (49 mg, 0.08 mmol) was dissolved in MeOH (3 mL). To the mixture was then added NaHCO₃ (20 mg), followed by MeI (0.1 ml, 1.58 mmol) and the mixture was stirred at room temperature for 4 h. The volatile was removed under reduced pressure and the residue was re-dissolved in MeOH. The precipitate was filtered off and the filtrate was purified by Gilson HPLC purification to yield the title compound a white foam.

¹H NMR (400 MHz, METHANOL-d4) Shift 7.30-7.37 (m, 2H), 7.24-7.29 (m, 1H), 7.11 (d, J=8.59 Hz, 2H), 6.83 (d, J=8.59 Hz, 2H), 4.08 (d, J=9.09 Hz, 1H), 3.97-4.05 (m, 4H), 3.87 (br d, J=11.12 Hz, 1H), 3.68 (br dd, J=5.31, 11.87 Hz, 1H), 3.46-3.58 (m, 4H), 3.35-3.46 (m, 5H), 3.26-3.29 (m, 1H), 3.05 (s, 3H), 2.20 (br s, 4H), 1.92-2.05 (m, 2H), 1.78-1.89 (m, 2H). m/z (MH⁺): 520.20.

### EXAMPLE 3

### Compound #15

### 2-(4-(2-chloro-5-((2S,3R,4R,5S,6R)-3,4,5-trihydroxy-6-(hydroxymethyl)tetrahydro-2H-pyran-2-yl)benzyl)phenoxy)-N-(2-(4-(2-chloro-4-((2S,3R,4R,5S,6R)-3,4,5-trihydroxy-6-(hydroxymethyl)tetrahydro-2H-pyran-2-yl)benzyl)phenoxy)ethyl)-N,N-dimethylethan-1-aminium trifluoroacetate

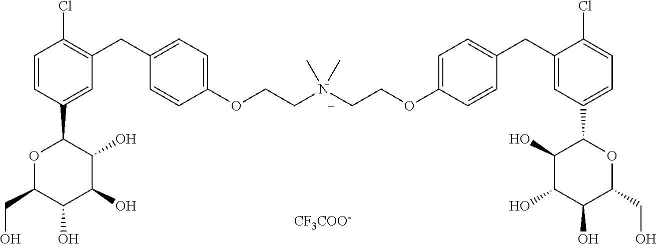

A 2-dram vial was charged with (2R,3R,4R,5S,6S)-2-(acetoxymethyl)-6-(4-chloro-3-(4-hydroxybenzyl)phenyl)tetrahydro-2H-pyran-3,4,5-triyl triacetate (48.6 mg, 0.089 mmol), 2-chloro-N-(2-chloroethyl)-N-methylethan-1-amine hydrochloride (8.9 mg, 0.046 mmol) and Cs₂CO₃ (86.5 mg, 0.27 mmol). To the mixture was then added acetonitrile (1 mL) and the mixture was stirred at 65° C. for 2 h. The reaction mixture was then treated with MeI (0.05 ml, 0.8 mmol) and then stirred at room temperature for 1 h. The solvent was removed under reduced pressure and the residue was re-dissolved in MeOH (1 ml), the mixture treated NaOMe (25% solution in MeOH, 0.05 mL) and the resulting mixture maintained at room temperature for 2 h. The reaction mixture was then neutralized with 1N HCl and then subjected to Gilson HPLC purification (15 min gradient time, 10% acetonitrile to 90% acetonitrile). The pure fractions were combined and lyophilized to yield 2-(4-(2-chloro-5-((2S,3R,4R,5S,6R)-3,4,5-trihydroxy-6-(hydroxymethyl)tetrahydro-2H-pyran-2-yl)benzyl)phenoxy)-N-(2-(4-(2-chloro-4-((2S,3R,4R,5S,6R)-3,4,5-trihydroxy-6-(hydroxymethyl)tetrahydro-2H-pyran-2-yl)benzyl)phenoxy)ethyl)-N,N-dimethylethan-1-aminium trifluoroacetate as a white solid.

¹H NMR (400 MHz, MeOH) Shift 7.33 (s, 4H), 7.26-7.31 (m, 2H), 7.16 (d, J=8.59 Hz, 4H), 6.90 (d, J=8.59 Hz, 4H), 4.49 (br s, 4H), 4.07 (dd, J=8.84, 13.89 Hz, 6H), 3.92-3.97 (m, 4H), 3.84-3.90 (m, 2H), 3.68 (dd, J=5.31, 11.87 Hz, 2H), 3.36-3.47 (m, 6H), 3.29-3.33 (m, 12H), 3.25-3.28 (m, 2H). m/z (M+): 858.30.

### EXAMPLE 4

### Compound #3

### (3-((4-(2-chloro-4-((2S,3R,4R,5S,6R)-3,4,5-trihydroxy-6-(hydroxymethyl)tetrahydro-2H-pyran-2-yl)benzyl)phenoxy)methyl)benzoyl)-L-glutamic acid

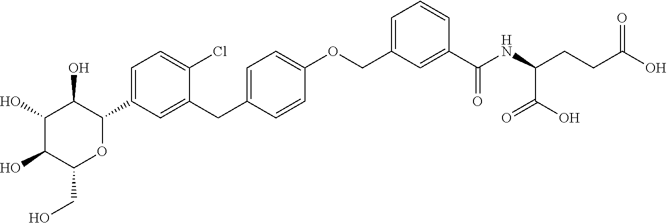 [SMILES: O=C(O)CC[C@H](NC(=O)c1cccc(COc2ccc(Cc3cc([C@@H]4O[C@H](CO)[C@@H](O)[C@H](O)[C@H]4O)ccc3Cl)cc2)c1)C(=O)O]

### Step 1: 3-(chloromethyl)benzoyl chloride

To a suspension of 3-(chloromethyl)benzoic acid (1 g, 5.86 mmol) in DCM (30 ml) was added oxalyl chloride (0.63 ml, 7.03 mmol), followed by 2 drops of DMF. The resulting mixture was stirred at room temperature for 1 h, then the volatiles were removed under reduced pressure to yield 3-(chloromethyl)benzoyl chloride, which was used for the next step reaction without further purification.

### Step 2: Dimethyl (3-(chloromethyl)benzoyl)-L-glutamate

To a mixture of 3-(chloromethyl)benzoyl chloride and L-glutamic acid dimethyl ester (1.24 g, 5.862 mmol) was added DCM (30 mL), followed by dropwise addition of triethylamine (2.44 ml, 17.59 mmol) and the resulting mixture was stirred at room temperature for 16 h, then quenched with 1N HCl (30 ml). The organic layer was separated and the aqueous layer was extracted with EtOAc. The combined organic layer was dried with Na₂SO₄. The resulting solid was filtered and the filtrate was concentrated. The residue was purified by flash column chromatography on silica gel (12 g, EtOAc/heptane: 0>>>10%>>>30%) to yield dimethyl (3-(chloromethyl)benzoyl)-L-glutamate as a colorless syrup. m/z (MH+): 328.0.

### Step 3: Dimethyl (3-((4-(2-chloro-5-((2S,3S,4R,5R,6R)-3,4,5-triacetoxy-6-(acetoxymethyl)tetrahydro-2H-pyran-2-yl)benzyl)phenoxy)methyl)benzoyl)-L-glutamate

To a mixture of (2R,3R,4R,5S,6S)-2-(acetoxymethyl)-6-(3-chloro-4-(4-hydroxybenzyl)phenyl)tetrahydro-2H-pyran-3,4,5-triyltriacetate (50 mg, 0.091 mmol), Cs₂CO₃ (89 mg, 0.27 mmol), and dimethyl (3-(chloromethyl)benzoyl)-L-glutamate (32.8 mg, 0.10 mmol) was added anhydrous DMF (1 mL) and the resulting mixture was stirred at room temperature for 2 h. The reaction mixture was monitored by LC/MS. The reaction mixture was diluted with EtOAc, partitioned between EtOAc and 1N HCl. The organic layers were combined, dried with Na₂SO₄. The precipitate was filtered off and the filtrate was concentrated under reduced pressure. The residue was purified by flash column chromatography on silica gel (40 g, EtOAc/heptane: 0>>>60%) to yield dimethyl (3-((4-(2-chloro-5-((2S,3S,4R,5R,6R)-3,4,5-triacetoxy-6-(acetoxymethyl)tetrahydro-2H-pyran-2-yl)benzyl)phenoxy)methyl)benzoyl)-L-glutamate as a white solid.

### Step 4: (3-((4-(2-chloro-54(2S,3R,4R,5S,6R)-3,4,5-trihydroxy-6-(hydroxymethyl)tetrahydro-2H-pyran-2-yl)benzyl)phenoxy)methyl)benzoyl)-L-glutamic acid

To dimethyl (3-((4-(2-chloro-4-((2S,3S,4R,5R,6R)-3,4,5-triacetoxy-6-(acetoxymethyl)tetrahydro-2H-pyran-2-yl)benzyl)phenoxy)methyl)benzoyl)-L-glutamate (56 mg, 0.067 mmol) was added MeOH (10 mL), followed NaOMe (25% in MeOH, 3 drops) and the resulting mixture was stirred at room temperature for 16 h. To the resulting mixture was then added 50% NaOH aqueous solution (10 drops) and the mixture was briefly heated with a heat gun, then stirred at room temperature for 30 min. The mixture was concentrated under reduced pressure and the residue was diluted with 1N HCl (20 ml), then extracted with EtOAc three times. The organic layers were combined and washed with brine, dried with Na₂SO₄. The drying agent was filtered off and the volatiles were removed under reduced pressure to yield (3-((4-(2-chloro-5-((2S,3R,4R,5S,6R)-3,4,5-trihydroxy-6-(hydroxymethyl)tetrahydro-2H-pyran-2-yl)benzyl)phenoxy)methyl)benzoyl)-L-glutamic acid as a white foam.

¹H NMR (400 MHz, METHANOL-d4) Shift 7.91-7.97 (m, 1H), 7.75-7.84 (m, 1H), 7.59-7.66 (m, 1H), 7.48 (s, 1H), 7.23-7.37 (m, 3H), 7.12 (br d, J=8.59 Hz, 2H), 6.91 (br d, J=8.59 Hz, 2H), 5.11 (s, 2H), 4.63 (dd, J=4.80, 9.35 Hz, 1H), 3.98-4.11 (m, 3H), 3.86 (br d, J=12.13 Hz, 1H), 3.63-3.72 (m, 1H), 3.35-3.49 (m, 4H), 2.42-2.53 (m, 2H), 2.24-2.36 (m, 1H), 2.03-2.16 (m, 1H). m/z (MH+): 644.25.

### EXAMPLE 5

### Compound #19

### N-(2-(4-(5-(2-chloro-5-((2S,3R,4R,5S,6R)-3,4,5-trihydroxy-6-(hydroxymethyl)tetrahydro-2H-pyran-2-yl)benzyl)thiophen-2-yl)phenoxy)ethyl)-3-(4-(5-(2-chloro-5-((2S,3R,4R,5S,6R)-3,4,5-trihydroxy-6-(hydroxymethyl)tetrahydro-2H-pyran-2-yl)benzyl)thiophen-2-yl)phenyl)-N,N-dimethylpropan-1-aminium trifluoroacetate

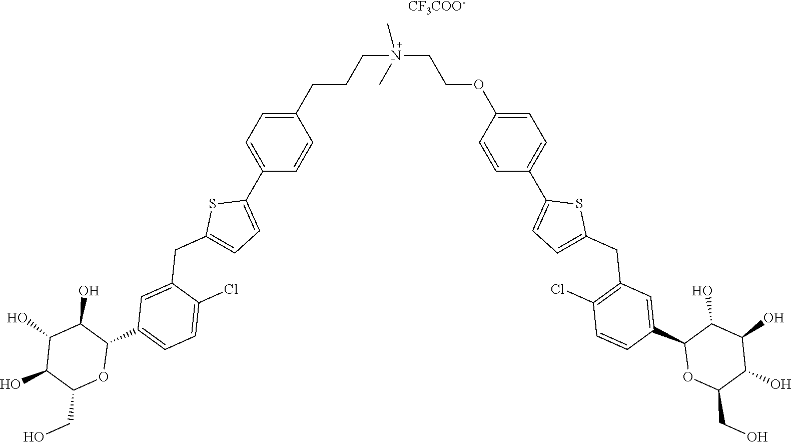

### Step 1: 2-Chloro-5-iodobenzoyl chloride

To a suspension of 5-Iodo-2-chlorobenzoic acid (5.07 g, 17.9 mmol) in DCM (35 mL) was added oxalyl chloride (1.95 mL) and the mixture was stirred at room temperature for 5 min. To the mixture was then added DMF (3 drops) and the resulting mixture was stirred at room temperature for 2 h. The volatiles were removed under reduced pressure to yield a colorless solid, which was used for the next step reaction without further purification.

### Step 2: (2-Chloro-5-iodophenyl)(thiophen-2-yl)methanone

2-Chloro-5-iodobenzoyl chloride (5.4 g, 17.9 mmol) was dissolved in DCM (50 mL). To the solution were then added thiophene (1584 mg, 18.83 mmol) and then AlCl₃ (3.61 g, 27.04 mmol) portion wise so that the temperature did not exceed −4° C. The resulting mixture was then stirred at 5° C. for 1 h, the mixture was poured into ice water and extracted with CHCl₃ three times. The combined organic layers were washed with 1M hydrochloric acid, water and brine, then dried over MgSO₄ and concentrated to yield a white solid which was used for the next step reaction without further purification.

### Step 3: 2-(2-Chloro-5-iodobenzyl)thiophene

(2-Chloro-5-iodophenyl)(thiophen-2-yl)methanone (6.25 g, 17.9 mmol) was dissolved in MeOH (80 mL) and DCM (30 mL). To the mixture was then added NaBH₄ (950.2 mg, 25.1 mmol) in 4 portions and the resulting mixture was stirred at room temperature for 40 min. The mixture was then concentrated under reduced pressure and the residue was diluted with EtOAc, washed with 1N HCl three times, dried with Na₂SO₄. The drying agent was filtered off and the filtrate was concentrated under reduced pressure to yield a brown oil. The brown oil was dissolved in DCM (80 mL), and the mixture cooled to 0° C. To the mixture was then added triethylsilane (6 ml, 37.5 mmol), followed by BF₃.Et₂O. The reaction mixture was stirred at 0° C. for 45 min. The reaction was quenched with aqueous NaHCO₃ solution and the organic layer was separated. The aqueous layer was extracted with DCM three times and the combined DCM extracts were washed with brine, dried with Na₂SO₄. The insoluble solid was filtered off and the filtrate was concentrated. The residue was purified by flash column chromatography on silica gel (120 g, EtOAc/heptane: 0>>>10) to yield 2-(2-chloro-5-iodobenzyl)thiophene as a colorless oil.

### Step 4: (3R,4S,5S,6R)-2-(4-Chloro-3-(thiophen-2-ylmethyl)phenyl)-6-(hydroxymethyl)-2-methoxytetrahydro-2H-pyran-3,4,5-triol

2-(2-Chloro-5-iodobenzyl)thiophene (2.93 g, 8.76 mmol) was dissolved in anhydrous THF (5 mL), and the mixture cooled to −78° C. in a dry ice-acetone bath. The flask was evacuated, re-filled with argon and this process was repeated three times. To the mixture was then added i-PrMgCl.LiCl (7.2 ml, 9.37 mmol) dropwise and the resulting mixture was stirred at −78° C. for 2 h. The progress of the metal-halogen exchange was monitored by LC/MS and the exchange was deemed finished around 2 h time point. A solution of (3R,4S,5R,6R)-3,4,5-tris((trimethylsilyl)oxy)-6-(((trimethylsilyl)oxy)methyl)tetrahydro-2H-pyran-2-one (4.09 g, 8.76 mmol) in THF (7 mL) was added at −78° C. and the reaction mixture was warmed to 0° C. for 3 h. A solution of MeSO₃H (1.07 g, 11.12 mmol) in MeOH (18 mL) was added to the above mixture slowly at 0° C. and the resulting mixture was stirred at 0° C. for 10 min, then warmed to room temperature and held, with stirring for 16 h. The mixture was then diluted with saturated aqueous NaHCO₃ (30 ml) and extracted with EtOAc four times and the combined extracts were washed with brine, dried with Na₂SO₄. The insoluble solid was filtered off and the filtrate was concentrated under reduced pressure to yield (3R,4S,5S,6R)-2-(4-chloro-3-(thiophen-2-ylmethyl)phenyl)-6-(hydroxymethyl)-2-methoxytetrahydro-2H-pyran-3,4,5-triol as a light orange foam. m/z (M+Na): 423.0.

### Step 5: (2S,3R,4R,5S,6R)-2-(4-Chloro-3-(thiophen-2-ylmethyl)phenyl)-6-(hydroxymethyl)tetrahydro-2H-pyran-3,4,5-triol

(3R,4S,5S,6R)-2-(4-chloro-3-(thiophen-2-ylmethyl)phenyl)-6-(hydroxymethyl)-2-methoxytetrahydro-2H-pyran-3,4,5-triol (3.51 g, 8.76 mmol) was dissolved in DCM/acetonitrile (2:3 v/v) (50 mL) and the solution was cooled to 0° C. To the mixture was then added triethylsilane (3.08 ml, 19.3 mmol), followed by BF₃.Et₂O dropwise and the resulting mixture was stirred at 0° C. for 1 h. The reaction mixture was quenched with aqueous NaHCO₃, extracted with EtOAc three times and the combined extracts were dried with Na₂SO₄. The solid was filtered off and the filtrate was concentrated under reduced pressure. The residue was purified by flash column chromatography on silica gel (40 g, EtOAc/heptane: 0>>>100%, then 1% MeOH in EtOAc) to yield (2S,3R,4R,5S,6R)-2-(4-chloro-3-(thiophen-2-ylmethyl)phenyl)-6-(hydroxymethyl)tetrahydro-2H-pyran-3,4,5-triol as a light orange foam.

### Step 6: (2R,3R,4R,5S,6S)-2-(Acetoxymethyl)-6-(4-chloro-3-(thiophen-2-ylmethyl)phenyl)tetrahydro-2H-pyran-3,4,5-triyl triacetate

To a solution of (2S,3R,4R,5S,6R)-2-(4-chloro-3-(thiophen-2-ylmethyl)phenyl)-6-(hydroxymethyl)tetrahydro-2H-pyran-3,4,5-triol (1.8 g, 4.85 mmol) and pyridine (3.91 ml, 48.54 mmol) in DCM (45 ml) was added DMAP (30 mg, 0.24 mmol), followed by acetic anhydride (4.6 ml, 48.54 mmol), and the resulting mixture was stirred at room temperature for 16 h. Saturated NaHCO₃ solution (20 ml) was added and the mixture was stirred at room temperature for 30 min. The organic layer was then separated, and the aqueous layer was extracted with DCM three times (25 ml each time). The combined DCM extracts were washed with 1N HCl three times (15 ml each time), dried with Na₂SO₄ and the insoluble solid was filtered off. The filtrate was concentrated under reduced pressure to yield a white foam, which was dissolved in hot EtOH. A white solid was crystallized from EtOH while being cooled to −40° C. The solid was filtered off and then dried in vacuo to yield (2R,3R,4R,5S,6S)-2-(acetoxymethyl)-6-(4-chloro-3-(thiophen-2-ylmethyl)phenyl)tetrahydro-2H-pyran-3,4,5-triyltriacetate as a white solid. ¹H NMR (400 MHz, CHLOROFORM-d) δ 7.36 (s, 1H), 7.19-7.25 (m, 1H), 7.13-7.19 (m, 2H), 6.93 (dd, J=3.54, 5.05 Hz, 1H), 6.79 (d, J=3.03 Hz, 1H), 5.26-5.33 (m, 1H), 5.17-5.24 (m, 1H), 5.06 (t, J=9.60 Hz, 1H), 4.34 (d, J=10.11 Hz, 1H), 4.19-4.31 (m, 3H), 4.12-4.18 (m, 1H), 3.77-3.85 (m, 1H), 2.08 (s, 3H), 2.05 (s, 3H), 1.99 (s, 3H), 1.74 (s, 3H).

### Step 7: (2R,3R,4R,5S,6S)-2-(Acetoxymethyl)-6-(3-((5-bromothiophen-2-yl)methyl)-4-chlorophenyl)tetrahydro-2H-pyran-3,4,5-triyl triacetate

(2R,3R,4R,5S,6S)-2-(acetoxymethyl)-6-(4-chloro-3-(thiophen-2-ylmethyl)phenyl)tetrahydro-2H-pyran-3,4,5-triyltriacetate (768.1 mg, 1.43 mmol) was dissolved in acetonitrile (20 mL). To the mixture was then added NBS (266.3 mg, 1.50 mmol) and the mixture was stirred at room temperature for 1 h. The resulting mixture was diluted with aqueous Na₂S₂O₃ solution, extracted with EtOAc three times. The combined extracts were dried with Na₂SO₄. The solid was filtered off and the filtrate was concentrated under reduced pressure to yield a light orange solid, which was purified by flash column chromatography on silica gel (12 g, EtOAc/heptane: 0>>>10%>>>30%) to yield (2R,3R,4R,5S,6S)-2-(acetoxymethyl)-6-(3-((5-bromothiophen-2-yl)methyl)-4-chlorophenyl)tetrahydro-2H-pyran-3,4,5-triyltriacetate as a white solid. ¹H NMR (400 MHz, CHLOROFORM-d) δ 7.37 (d, J=8.08 Hz, 1 H), 7.20-7.25 (m, 1H), 7.18 (d, J=1.52 Hz, 1H), 6.86 (d, J=3.54 Hz, 1H), 6.54 (d, J=3.54 Hz, 1H), 5.27-5.34 (m, 1H), 5.17-5.25 (m, 1H), 5.02-5.11 (m, 1H), 4.32-4.38 (m, 1H), 4.24-4.31 (m, 1H), 4.09-4.23 (m, 3H), 3.82 (ddd, J=2.27, 4.67, 9.73 Hz, 1H), 2.09 (s, 3H), 2.06 (s, 3H), 2.00 (s, 3H), 1.77 (s, 3H). m/z (M+Na): 640.95.

### Step 8: (2R,3R,4R,5S,6S)-2-(Acetoxymethyl)-6-(4-chloro-3-((5-(4-((tetrahydro-2H-pyran-2-yl)oxy)phenyl)thiophen-2-yl)methyl)phenyl)tetrahydro-2H-pyran-3,4,5-triyl triacetate

A 40 ml vial was charged with (2R,3R,4R,5S,6S)-2-(acetoxymethyl)-6-(3-((5-bromothiophen-2-yl)methyl)-4-chlorophenyl)tetrahydro-2H-pyran-3,4,5-triyl triacetate (568.3 mg, 0.92 mmol), (4-((tetrahydro-2H-pyran-2-yl)oxy)phenyl)boronic acid (408.5 mg, 1.84 mmol), and PdCl₂(dppf)₂ (33.6 mg, 0.046 mmol) and the vial was capped with a rubber stopper. The vial was evacuated, refilled with argon and this process was repeated twice. To the mixture was then added toluene (10 ml), followed by 2M K₂CO₃ (1.61 mL) solution. The vial was then replaced with a Telfon cap and the mixture was stirred at 100° C. for 3 h. The mixture was diluted with EtOAc (15 ml), dried with Na₂SO₄ and the insoluble solid/catalyst was filtered off. The filtrate was concentrated. The residue was purified by flash column chromatography on silica gel (12 g, EtOAc/heptane: 0>>>50%>>>100%) to yield (2R,3R,4R,5S,6S)-2-(Acetoxymethyl)-6-(4-chloro-3-((5-(4-((tetrahydro-2H-pyran-2-yl)oxy)phenyl)thiophen-2-yl)methyl)phenyl)tetrahydro-2H-pyran-3,4,5-triyl triacetate as a light orange solid.

### Step 9: (2R,3R,4R,5S,6S)-2-(acetoxymethyl)-6-(4-chloro-3-((5-(4-hydroxyphenyl)thiophen-2-yl)methyl)phenyl)tetrahydro-2H-pyran-3,4,5-triyl triacetate

A 100 ml round bottom flask as charged with (2R,3R,4R,5S,6S)-2-(Acetoxymethyl)-6-(4-chloro-34(5-(4-((tetrahydro-2H-pyran-2-yl)oxy)phenyl)thiophen-2-yl)methyl)phenyl)tetrahydro-2H-pyran-3,4,5-triyl triacetate (620 mg, 0.87 mmol). To the mixture was then added THF (18 mL), followed by 1N HCl (6 mL). The mixture became a little cloudy, MeOH (1 mL) was added and the resulting mixture was stirred at room temperature for 2 h. The progress of the hydrolysis was monitored by TLC. After 2h, the mixture was concentrated under reduced pressure and the residue was partitioned between EtOAc and water. The aqueous layer was extracted with EtOAc three times and the combined EtOAc extracts were washed with brine, dried with Na₂SO₄. The drying agent was filtered off and the filtrate was concentrated to yield a brown solid, which was further purified by flash column chromatography on silica gel (40 g, EtOAc/heptane: 0>>>30%>>>50%) to provide (2R,3R,4R,5S,6S)-2-(acetoxymethyl)-6-(4-chloro-3-((5-(4-hydroxyphenyl)thiophen-2-yl)methyl)phenyl)tetrahydro-2H-pyran-3,4,5-triyl triacetate as a white solid.

### Step 10: (2R,3R,4R,5S,6S)-2-(Acetoxymethyl)-6-(4-chloro-3-((5-(4-(24(3-(4-(5-(2-chloro-5-((25,3S,4R,5R,6R)-3,4,5-triacetoxy-6-(acetoxymethyl)tetrahydro-2H-pyran-2-yl)benzyl)thiophen-2-yl)phenyl)propyl)(methyl)amino)ethoxy)phenyl)thiophen-2-yl)methyl)phenyl)tetrahydro-2H-pyran-3,4,5-triyltriacetate

A 2-dram vial was charged with (2R,3R,4R,5S,6S)-2-(acetoxymethyl)-6-(4-chloro-3-((5-(4-hydroxyphenyl)thiophen-2-yl)methyl)phenyl)tetrahydro-2H-pyran-3,4,5-triyltriacetate (56.1 mg, 0.089 mmol), 2-chloro-N-(2-chloroethyl)-N-methylethan-1-amine hydrochloride (8.9 mg, 0.046 mmol) and Cs₂CO₃ (86.9 mg, 0.27 mmol). To the mixture was then added acetonitrile (1 mL) and the mixture was stirred at 65° C. for 2 h, then was used for the next step reaction directly.

### Step 11: N-(2-(4-(5-(2-Chloro-5-((2S,3R,4R,5S,6R)-3,4,5-trihydroxy-6-(hydroxymethyl)tetrahydro-2H-pyran-2-yl)benzyl)thiophen-2-yl)phenoxy)ethyl)-3-(4-(5-(2-chloro-5-((2S,3R,4R,5S,6R)-3,4,5-trihydroxy-6-(hydroxymethyl)tetrahydro-2H-pyran-2-yl)benzyl)thiophen-2-yl)phenyl)-N,N-dimethylpropan-1-aminium trifluoroacetate

(2R,3R,4R,5S,6S)-2-(acetoxymethyl)-6-(4-chloro-3-((5-(4-(2-((3-(4-(5-(2-chloro-5-((2S,3S,4R,5R,6R)-3,4,5-triacetoxy-6-(acetoxymethyl)tetrahydro-2H-pyran-2-yl)benzyl)thiophen-2-yl)phenyl)propyl)(methyl)amino)ethoxy)phenyl)thiophen-2-yl)methyl)phenyl)tetrahydro-2H-pyran-3,4,5-triyl triacetate (59.9 mg, 0.045 mmol) in acetonitrile (1 mL) was treated with Mel (0.05 ml, 0.8 mmol) and the mixture was stirred at room temperature for 1 h. The volatiles were removed under reduced pressure and the residue was re-dissolved in MeOH (1 ml), treated with NaOMe (25% solution in MeOH, 0.05 mL) and the mixture was left at room temperature overnight. The resulting mixture was neutralized with 1N HCl and then subjected to Gilson HPLC purification (12 min gradient time, 10% acetonitrile>>>95% acetonitrile) to yield N-(2-(4-(5-(2-Chloro-5-((2S,3R,4R,5S,6R)-3,4,5-trihydroxy-6-(hydroxymethyl)tetrahydro-2H-pyran-2-yl)benzyl)thiophen-2-yl)phenoxy)ethyl)-3-(4-(5-(2-chloro-5-((2S,3R,4R,5S,6R)-3,4,5-trihydroxy-6-(hydroxymethyl)tetrahydro-2H-pyran-2-yl)benzyl)thiophen-2-yl)phenyl)-N,N-dimethylpropan-1-aminium trifluoroacetate as a white solid.

¹H NMR (400 MHz, MeOH) Shift 7.48-7.55 (m, 4H), 7.45 (d, J=1.52 Hz, 2H), 7.36-7.42 (m, 2H), 7.33 (br d, J=2.02 Hz, 2H), 7.06 (d, J=3.54 Hz, 2H), 6.98 (br d, J=9.09 Hz, 4H), 6.78 (br s, 2H), 4.55 (br d, J=2.02 Hz, 4H), 4.20-4.33 (m, 4H), 4.12 (br d, J=9.60 Hz, 2H), 3.98 (br d, J=4.04 Hz, 4H), 3.84-3.92 (m, 2H), 3.65-3.75 (m, 2H), 3.38-3.50 (m, 6H), 3.35 (s, 6H), 3.30 (br s, 2H). m/z (M+): 1022.2.

### EXAMPLE 6

### Compound #2

### 3-(4-(2-chloro-5-((2S,3R,4R,5S,6R)-3,4,5-trihydroxy-6-(hydroxymethyl)tetrahydro-2H-pyran-2-yl)benzyl)phenoxy)propane-1-sulfonic acid

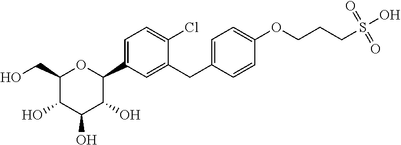 [SMILES: O=S(=O)(O)CCCOc1ccc(Cc2cc([C@@H]3O[C@H](CO)[C@@H](O)[C@H](O)[C@H]3O)ccc2Cl)cc1]

To a mixture of (2R,3R,4R,5S,6S)-2-(acetoxymethyl)-6-(4-chloro-3-(4-hydroxybenzyl)phenyl)tetrahydro-2H-pyran-3,4,5-triyltriacetate (60 mg, 0.11 mmol) and 1,2-oxathiolane 2,2-dioxide (20 mg, 0.16 mmol) in acetonitrile (1 mL) was added Cs₂CO₃ (71.2 mg, 0.22 mmol) and the resulting mixture was stirred at 70° C. for 1 h. To the resulting mixture was then added methanol (1 mL), followed by addition of NaOMe (25% in MeOH, 1mL) and the resulting mixture was stirred at room temperature for 1 h. The resulting precipitate was filtered off and the filtrate was subjected to Gilson HPLC purification (12 min gradient time, 10% acetonitrile with 0.05% TFA to 95% acetonitrile with 0.05% TFA). combined fractions were concentrated under reduced pressure to yield 3-(4-(2-chloro-5-((2S,3R,4R,5S,6R)-3,4,5-trihydroxy-6-(hydroxymethyl)tetrahydro-2H-pyran-2-yl)benzyl)phenoxy)propane-1-sulfonic acid as a white solid.

¹H NMR (400 MHz, MeOH) Shift 7.29-7.36 (m, 2H), 7.24-7.29 (m, 1H), 7.05-7.14 (m, 2H), 6.83 (s, 2H), 3.96-4.11 (m, 5H), 3.80-3.92 (m, 1H), 3.64-3.73 (m, 1H), 3.36-3.46 (m, 3H), 3.24-3.28 (m, 1H), 2.94-3.02 (m, 2H), 2.17-2.26 (m, 2H). m/z(M+18): 520.2.

### EXAMPLE 7

### Compound of Formula (III)

### (2R,2′R,2″R,3S,3′S,3″S,4R,4′R,4″R,5R,5′R,5″R,6S,6′S,6″S)-6,6′,6″-((((((5-((4-(2-chloro-5-((2R,3R,4R,5S,6R)-3,4,5-trihydroxy-6-(hydroxymethyl)tetrahydro-2H-pyran-2-yl)benzyl)phenoxy)methyl)benzene-1,2,4-triyl)tris(methylene))tris(oxy))tris(benzene-4,1-diyl))tris(methylene))tris(4-chlorobenzene-3,1-diyl))tris(2-(hydroxymethyl)tetrahydro-2H-pyran-3,4,5-triol)

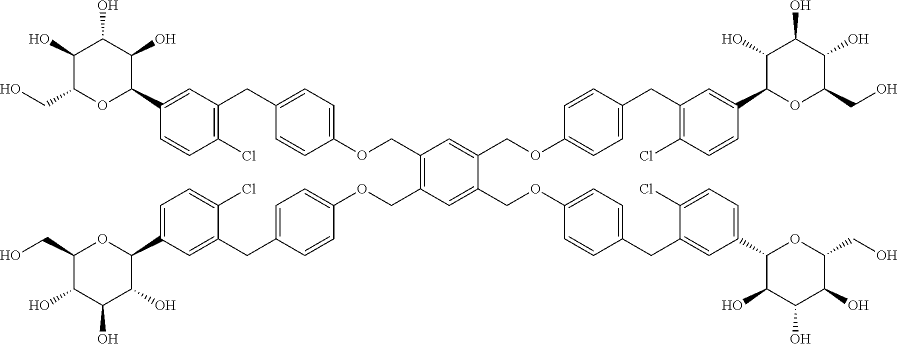 [SMILES: OC[C@H]1O[C@H](c2ccc(Cl)c(Cc3ccc(OCc4cc(COc5ccc(Cc6cc([C@@H]7O[C@H](CO)[C@@H](O)[C@H](O)[C@H]7O)ccc6Cl)cc5)c(COc5ccc(Cc6cc([C@@H]7O[C@H](CO)[C@@H](O)[C@H](O)[C@H]7O)ccc6Cl)cc5)cc4COc4ccc(Cc5cc([C@@H]6O[C@H](CO)[C@@H](O)[C@H](O)[C@H]6O)ccc5Cl)cc4)cc3)c2)[C@H](O)[C@@H](O)[C@@H]1O]

A 2-dram vial was charged with (2R,3R,4R,5S,6S)-2-(acetoxymethyl)-6-(4-chloro-3-(4-hydroxybenzyl)phenyl)tetrahydro-2H-pyran-3,4,5-triyl triacetate (76.1 mg, 0.14 mmol), 1,2,4,5-tetrakis(bromomethyl)benzene (15.6 mg, 0.035 mmol), Cs₂CO₃ (67.7 mg, 0.21 mmol) and acetonitrile (1 mL). The vial was sealed with a TFE cap and the resulting mixture was stirred at 65° C. for 1.5 h. To the resulting mixture was then added NaOMe (25% in MeOH, 50 μL), followed by MeOH (1 mL), and the mixture was stirred at 65° C. for 1 h. The resulting precipitate was filtered off and the filtrate was subjected to Gilson HPLC purification twice to yield (2R,2′R,2″R,3S,3′S,3″S,4R,4′R,4″R,5R,5′R,5″R,6S,6′S,6″S)-6,6′,6″-((((((5-((4-(2-chloro-5-((2R,3R,4R,5S,6R)-3,4,5-trihydroxy-6-(hydroxymethyl)tetrahydro-2H-pyran-2-yl)benzyl)phenoxy)methyl)benzene-1,2,4-triyl)tris(methylene))tris(oxy))tris(benzene-4,1-diyl))tris(methylene))tris(4-chlorobenzene-3,1-diyl))tris(2-(hydroxymethyl)tetrahydro-2H-pyran-3,4,5-triol) as a white solid.

¹H NMR (400 MHz, METHANOL-d4) Shift 7.53-7.59 (m, 2H), 7.29 (td, J=8.34, 16.67 Hz, 12H), 7.03 (br d, J=8.59 Hz, 8H), 6.79 (br d, J=8.59 Hz, 8H), 5.04 (s, 8H), 3.92-4.09 (m, 12H), 3.81-3.88 (m, 4H), 3.63-3.71 (m, 4H), 3.33-3.47 (m, 12H), 3.25-3.28 (m, 4H). m/z (M+Na): 1669.4

Additional representative compounds of formula (I) and formula (II) of the present invention were prepared as described herein (and, wherein said compounds contain one or more quaternary nitrogen atoms, prepared as their corresponding TFA salts), and their physical properties, more particularly LCMS and/or ¹H NMR, measured as listed in Table 3 below.

TABLE 3
¹H NMR and Mass Spec for Representative Compounds

| Cmpd No. | ¹H NMR/m/z Mass Spec |
|---|---|
| 1 | ¹H NMR (400 MHz, METHANOL-d4) Shift 7.30-7.36 (m, 2H), |
| | 7.24-7.29 (m, 1H), 7.10 (d, J = 8.59 Hz, 2H), 6.79-6.86 (m, 2H), |
| | 4.04-4.11 (m, 3H), 3.96-4.04 (m, 1H), 3.77-3.89 (m, 3H), 3.56- |
| | 3.71 (m, 10H), 3.50 (dd, J = 3.54, 5.56 Hz, 2H), 3.34-3.47 (m, |
| | 3H), 3.32 (s, 2H). m/z(MH+): 571.20. |
| 4 | ¹H NMR (400 MHz, METHANOL-d4) Shift 7.30-7.37 (m, 2H), |
| | 7.25-7.30 (m, 1H), 7.12 (d, J = 8.59 Hz, 2H), 6.84 (d, J = 8.59 Hz, |
| | 2H), 3.99-4.12 (m, 5H), 3.87 (d, J = 11.62 Hz, 1H), 3.68 (dd, |
| | J = 5.05, 12.13 Hz, 1H), 3.50-3.59 (m, 2H), 3.41-3.48 (m, 1H), |
| | 3.38 (br d, J = 7.07 Hz, 2H), 3.26-3.33 (m, 1H), 3.16 (s, 9H), 2.26 |
| | (qd, J = 5.45, 10.93 Hz, 2H). m/z (M+): 480.15. |
| 6 | ¹H NMR (400 MHz, METHANOL-d4) Shift 7.30-7.36 (m, 2H), |
| | 7.25-7.29 (m, 1H), 7.10 (d, J = 8.59 Hz, 2H), 6.81 (d, J = 8.59 Hz, |
| | 2H), 4.08 (d, J = 9.60 Hz, 1H), 3.95-4.05 (m, 4H), 3.84-3.90 (m, |
| | 1H), 3.65-3.72 (m, 1H), 3.42-3.57 (m, 4H), 3.33-3.40 (m, 5H), |
| | 3.28 (br s, 1H), 3.05 (s, 3H), 2.21 (br s, 4H), 1.81-1.92 (m, 4H), |
| | 1.57 (quin, J = 7.58 Hz, 2H). m/z(M+): 534.15. |
| 7 | ¹H NMR (400 MHz, METHANOL-d4) Shift 7.33 (s, 2H), 7.24- |
| | 7.30 (m, 1H), 7.12 (d, J = 8.59 Hz, 2H), 6.83 (d, J = 8.59 Hz, 2H), |
| | 4.12-4.17 (m, 2H), 4.00-4.10 (m, 3H), 3.93-3.99 (m, 2H), 3.81- |
| | 3.90 (m, 3H), 3.65-3.73 (m, 1H), 3.36-3.61 (m, 9H), 3.25-3.29 |
| | (m, 1H), 3.07 (s, 3H), 2.15 (br s, 4H). m/z (M+): 536.15. |
| 8 | ¹H NMR (400 MHz, METHANOL-d4) Shift 7.50-7.62 (m, 4H), |
| | 7.32 (s, 2H), 7.28 (s, 1H), 7.11 (d, J = 8.59 Hz, 2H), 6.88 (d, |
| | J = 8.59 Hz, 2H), 5.11 (s, 2H), 4.50 (s, 2H), 4.08 (d, J = 9.60 Hz, |
| | 1H), 3.95-4.05 (m, 2H), 3.87 (d, J = 11.12 Hz, 1H), 3.64-3.72 (m, |
| | 1H), 3.34-3.49 (m, 2H), 3.25-3.33 (m, 2H), 3.08 (s, 9H). m/z |
| | (M+): 542.20. |
| 9 | ¹H NMR (400 MHz, METHANOL-d4) Shift 7.52-7.63 (m, 4H), |
| | 7.29-7.36 (m, 2H), 7.28 (br d, J = 1.52 Hz, 1H), 7.11 (br d, |
| | J = 8.59 Hz, 2H), 6.88 (d, J = 8.59 Hz, 2H), 5.12 (s, 2H), 4.54 (s, |
| | 2H), 3.94-4.11 (m, 3H), 3.87 (br d, J = 11.62 Hz, 1H), 3.58-3.72 |
| | (m, 3H), 3.35-3.49 (m, 5H), 3.24-3.29 (m, 1H), 2.96 (s, 3H), |
| | 2.25 (br d, J = 4.55 Hz, 4H). m/z(M+): 568.20 |
| 11 | ¹H NMR (400 MHz, METHANOL-d4) Shift 7.29-7.35 (m, 4H), |
| | 7.24-7.29 (m, 2H), 7.08 (br d, J = 8.59 Hz, 4H), 6.80 (br d, |
| | J = 8.59 Hz, 4H), 3.95-4.11 (m, 10H), 3.82-3.90 (m, 2H), 3.76- |
| | 3.82 (m, 4H), 3.62-3.72 (m, 6H), 3.34-3.49 (m, 6H), 3.25-3.28 |
| | (m, 2H). m/z(MH+): 875.25 |
| 12 | ¹H NMR (400 MHz, MeOH) Shift 7.29-7.36 (m, 4H), 7.24-7.29 |
| | (m, 2H), 7.05-7.11 (m, 4H), 6.77-6.85 (m, 4H), 3.97-4.11 (m, |
| | 10H), 3.86 (dd, J = 1.77, 11.87 Hz, 2H), 3.75-3.81 (m, 4H), 3.59- |
| | 3.70 (m, 10H), 3.35-3.47 (m, 6H), 3.24-3.29 (m, 2H). m/z(MH+); |
| | 919.20 |
| 13 | ¹H NMR (400 MHz, METHANOL-d4) Shift 7.42 (s, 4H), 7.32 (br |
| | d, J = 4.55 Hz, 4H), 7.27 (br dd, J = 2.02, 8.08 Hz, 2H), 7.10 (br d, |
| | J = 8.08 Hz, 4H), 6.88 (br d, J = 8.59 Hz, 4H), 5.03 (s, 4H), 3.96- |
| | 4.11 (m, 6H), 3.86 (br d, J = 11.62 Hz, 2H), 3.68 (br dd, J = 4.80, |
| | 11.87 Hz, 2H), 3.34-3.49 (m, 6H), 3.26 (br s, 2H). m/z(MH+): 863.10 |
| 14 | ¹H NMR (400 MHz, METHANOL-d4) Shift 8.10-8.19 (m, 1H), |
| | 7.73 (d, J = 7.58 Hz, 2H), 7.30-7.36 (m, 4H), 7.28 (br d, J = 1.52 |
| | Hz, 2H), 7.14 (d, J = 8.59 Hz, 4H), 6.93 (d, J = 8.59 Hz, 4H), 5.26 |
| | (s, 4H), 3.96-4.11 (m, 6H), 3.81-3.91 (m, 2H), 3.68 (dd, J = 5.31, |
| | 11.87 Hz, 2H), 3.34-3.48 (m, 6H), 3.25-3.28 (m, 2H). m/z(MH+): 864.15 |
| 16 | ¹H NMR (400 MHz, MeOH) δ 7.52-7.67 (m, 8H), 7.24-7.38 (m, |
| | 6H), 7.08-7.16 (m, 4H), 6.85-6.94 (m, 4H), 5.13 (br s, 4H), 4.50- |
| | 4.62 (m, 4H), 3.98-4.12 (m, 6H), 3.86 (br d, J = 12.13 Hz, 2H), |
| | 3.64-3.73 (m, 2H), 3.35-3.50 (m, 14H), 3.27 (br s, 2H), 2.91- |
| | 3.06 (m, 6H), 1.91-2.08 (m, 5H), 1.45-1.79 (m, 5H). m/z (M/2 + 1): 582.0 |
| 17 | ¹H NMR (400 MHz, MeOH) δ 7.57 (br d, J = 6.57 Hz, 8H), 7.32 |
| | (d, J = 6.06 Hz, 6H), 7.11 (d, J = 8.59 Hz, 4H), 6.88 (d, J = 8.59 Hz, |
| | 4H), 5.08-5.16 (m, 4H), 4.48-4.62 (m, 4H), 4.05 (dd, J = 9.35, |
| | 19.45 Hz, 6H), 3.83-3.91 (m, 2H), 3.69 (br d, J = 5.05 Hz, 2H), |
| | 3.35-3.55 (m, 13H), 3.34 (s, 2H), 3.24-3.28 (m, 2H), 2.97-3.02 |
| | (m, 4H), 2.90-2.94 (m, 1H), 1.86-2.01 (m, 5H), 1.50-1.73 (m, |
| | 5H), 1.39 (br s, 4H). m/z(M/2): 595.3 |
| 18 | 1H NMR (400 MHz, MeOH) Shift 7.33 (s, 4H), 7.25-7.30 (m, |
| | 2H), 7.06-7.14 (m, 4H), 6.79 (s, 4H), 4.72 (br s, 2H), 4.08 (d, |
| | J = 9.09 Hz, 2H), 4.03 (br d, J = 8.08 Hz, 4H), 3.84-3.90 (m, 2H), |
| | 3.64-3.73 (m, 2H), 3.36-3.49 (m, 6H), 3.25-3.29 (m, 2H), 2.09- |
| | 2.22 (m, 4H). m/z(MH+); 903.2 |

### BIOLOGICAL EXAMPLE 1

### SGLT1 and SGLT2 Inhibition Assay (CHO K-1 and MDCK Cells)

The ability of the compounds of formula (I), formula (II) or formula (III) of the present invention to treat an SGLT-mediated condition was determined using the following procedures:

SGLT1 and SGLT2 were cloned in form of cDNA from human small intestine (Genbank M24847), and from human kidney (Genbank M95549), respectively. Subsequently, each full cDNA was subcloned into pcDNA with each construct's integrity verified through follow-on sequencing. To generate CHO-K1 or MDCK cells that stably express human SGLT1 or human SGLT2, CHO-K1 or MDCK cells were transfected using DMRIE-C reagent (Life Technologies, Gaithersburg, Md.). Transfected cells were then selected in the presence of 500 μg/ml of the Geneticin (G418 Cellgro Catalog No.30234-CI)

Individual clones were then characterized using the following cell-based assay for sodium-dependent glucose transport:

Inhibition of SGLT1 and SGLT2 activity was assessed in CHO K1 or MDCK cells stably expressing either human SGLT1 or SGLT2, using the SGLT specific glucose analog methyl-glucopyranoside (Sigma Catalog No. M-9376). Cells were plated (45,000 cells/well) in white wall 96-well plates (COSTAR, Cat #3903) for 24 hours in growth medium, then a final concentration of 10 mM Na-Butyrate (ALDRICH Cat #30341-0) was added. The cells were incubated for 24 hours. On the day of the assay, cells were rinsed and treated with test compounds (at concentrations of 0.001 μM to 10 μM) in assay buffer (50 mM HEPES, 20 mM Tris base, 5 mM KCl, 1 mM MgCl₂, 1 mM CaCl₂ and 137 mM NaCl, pH 7.4) for 10 minutes. Cells were then incubated with ¹⁴C-α-methyl-d-glucopyranoside (AMG, Amersham Catalog No. CFB 76), using 0.07 μCi per well in 500 μM AMG final concentration. The cells were incubated for 2 hours at 37° C. with 5% CO₂ and washed two times with ice-cold Phosphate Buffer Solution (Cellgro Catalog No. 21030-CV). The cells were then solubilized by adding 60 μl of MICROSCINT™ 20 and the Na-dependent ¹⁴C-AMG uptake was quantified by measuring radioactivity using scintillation counting. Plates were counted in a TopCount (Packard, Meriden, Conn.). Each experiment was repeated at least twice.

Representative compounds of the present invention were tested according to the procedures as described in Biological Example 1 above, with results as listed in Table 4 below. Results are reported as the %-inhibition or IC₅₀ value. Variability for the functional assay was typically within 20%. The %-inhibition or IC₅₀ data were derived from the best curve fit as listed in Table 4, below. When a listed compound was tested more than once, the average is listed in Table 4, below.

TABLE 4
SGLT1 and SGLT2 Activity

| Cmpd | CHO-K1 Cells | CHO-K1 Cells | MDCK Cells |
|---|---|---|---|
| No. | SGLT1% Inh @1 μM | SGLT2 IC₅₀ (nM) | SGLT2 IC₅₀ (μM) |
| 1 | 14.5% | 16.6 | 17 |
| 2 | 20% | 11.1 | |
| 3 | | 157 | |
| 4 | | 27.2 | |
| 5 | | 8.8 | |
| 6 | 43.1% | 3.0 | |
| 7 | 17.5% | 15.8 | |
| 9 | 54.6% | 2.4 | |
| 9 | 28% | 3.3 | |
| 10 | 1.491 μM (IC₅₀) | 7.6 | |
| 11 | | 20.8 | |
| 12 | | 9.6 | |
| 13 | | 32 | |
| 14 | 19% | 4.8 | 34 |
| 15 | | 121 | |
| 16 | | 4.0 | |
| 17 | | 5.0 | |
| 18 | | 377 | |
| 19 | | 24 | |
| (III) | | 33 | |

### FORMULATION EXAMPLE 1

### Solid, Oral Dosage Form—Prophetic Example

As a specific embodiment of an oral composition, 100 mg of the Compound #10, prepared as in Example 1 is formulated with sufficient finely divided lactose to provide a total amount of 580 to 590 mg to fill a size 0 hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

Throughout this application, various publications are cited. The disclosure of these publications is hereby incorporated by reference into this application to describe more fully the state of the art to which this invention pertains.

1. A compound of formula (I) 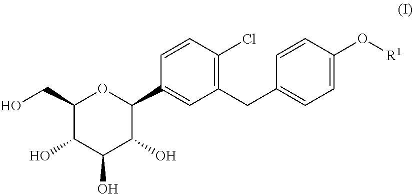
wherein
R¹ is selected from the group consisting of —CH₂CH₂—(OCH₂CH₂)ₐ—OCH₃, —CH₂CH₂CH₂—SO₃H, —(CH₂)_{b}—R², —CH₂CH₂—O—CH₂CH₂—R², 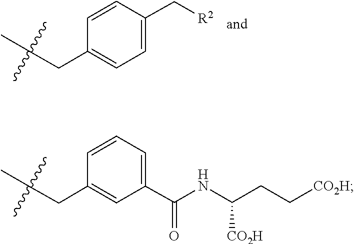
a is an integer from 2 to 12;
b is an integer from 2 to 6;
R² is selected from the group consisting of —N(CH₃)₃, 1-methyl-azetidin-1-yl, 1-methyl-pyrrolidin-1-yl, 1-methyl-piperidin-1-yl and 1-methyl-piperazin-1-yl;
or a pharmaceutically acceptable salt thereof.

2. The compound of claim 1, wherein
R¹ is selected from the group consisting of —CH₂CH₂—(OCH₂CH₂)ₐ—OCH₃, —CH₂CH₂CH₂—SO₃H, —(CH₂)_{b}—R², —CH₂CH₂—O—CH₂CH₂—R², 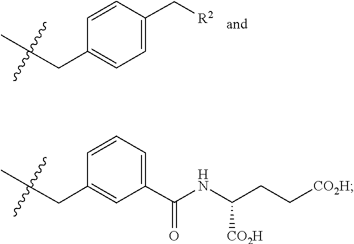
a is an integer from 2 to 6;
b is an integer from 2 to 5;
R² is selected from the group consisting of —N(CH₃)₃, 1-methyl-azetidin-1-yl, 1-methyl-pyrrolidin-1-yl and 1-methyl-piperidin-1-yl;
or a pharmaceutically acceptable salt thereof.

3. The compound of claim 1, wherein
R¹ is selected from the group consisting of —CH₂CH₂—(OCH₂CH₂)₃—OCH₃, —CH₂CH₂CH₂—SO₃H, —CH₂CH₂CH₂—N⁺(CH₃)₃, 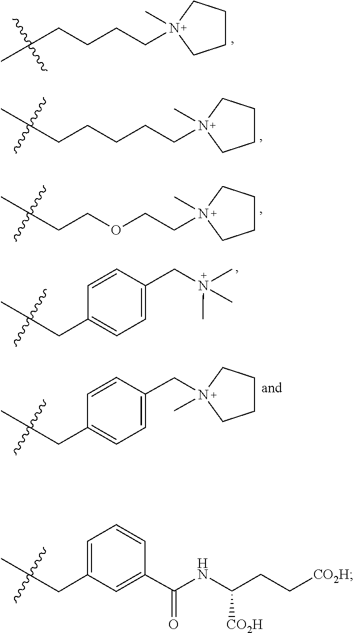
or a pharmaceutically acceptable salt thereof.

4. The compound of claim 1, wherein R¹ is selected from the group consisting of —CH₂CH₂CH₂—SO₃H, 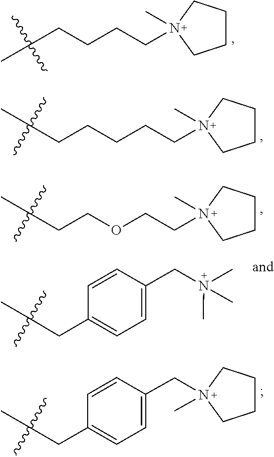
or a pharmaceutically acceptable salt thereof.

5. The compound of claim 1, wherein the compound is selected from the group consisting of
1-(4-(4-(2-chloro-5-((2S,3R,4R,5S,6R)-3,4,5-trihydroxy-6-(hydroxymethyl)tetrahydro-2H-pyran-2-yl)benzyl)phenoxy)butyl)-1-methylpyrrolidin-1-ium trifluoroacetate;
1-(5-(4-(2-chloro-5-((2S,3R,4R,5S,6R)-3,4,5-trihydroxy-6-(hydroxymethyl)tetrahydro-2H-pyran-2-yl)benzyl)phenoxy)pentyl)-1-methylpyrrolidin-1-ium trifluoroacetate;
1-(4-((4-(2-chloro-5-((2S,3R,4R,5S,6R)-3,4,5-trihydroxy-6-(hydroxymethyl)tetrahydro-2H-pyran-2-yl)benzyl)phenoxy)methyl)phenyl)-N,N,N-trimethylmethanaminium trifluoroacetate; and
1-(4-((4-(2-chloro-5-((2S,3R,4R,5S,6R)-3,4,5-trihydroxy-6-(hydroxymethyl)tetrahydro-2H-pyran-2-yl)benzyl)phenoxy)methyl)benzyl)-1-methylpyrrolidin-1-ium trifluoroacetate.

6. A pharmaceutical composition comprising a pharmaceutically acceptable carrier and the compound of claim 1.

7-8. (canceled)

9. A method of treating a disorder mediated by SGLT2 activity, comprising administering to a subject in need thereof a therapeutically effective amount of the compound of claim 1.

10. The method of claim 9, wherein the disorder mediated by SGLT2 activity is selected from the group consisting of impaired glucose tolerance (IGT), impaired fasting glucose (IFT), gestational diabetes, Type I diabetes mellitus, Type II diabetes mellitus, Syndrome X, obesity, nephropathy, neuropathy, retinopathy, hypertension, angina, atherosclerosis, heart disease, heart attack, ischemia, stroke, nerve damage or poor blood flow in the feet, non-alcoholic steatohepatitis (NASH), non-alcoholic fatty liver disease (NAFLD), liver fibrosis, cataracts, polycystic ovarian syndrome, irritable bowel syndrome, inflammation and cancer.

11. The method of claim 9, wherein the disorder mediated by SGLT2 activity is selected from the group consisting of impaired glucose tolerance, impaired fasting glucose, Type II Diabetes Mellitus, obesity, nephropathy, neuropathy, retinopathy, atherosclerosis, hypertension, heart disease, ischemia, stroke, non-alcoholic steatohepatitis (NASH), non-alcoholic fatty liver disease (NAFLD), liver fibrosis, cataracts, polycystic ovarian syndrome, irritable bowel disorder, inflammation, prostate cancer and pancreatic cancer.

12. The method of claim 9, wherein the disorder mediated by SGLT2 activity is selected from the group consisting of impaired glucose tolerance, impaired fasting glucose, Type II Diabetes Mellitus, obesity, nephropathy, neuropathy, retinopathy, atherosclerosis, hypertension, heart disease, ischemia, stroke, non-alcoholic steatohepatitis (NASH), non-alcoholic fatty liver disease (NAFLD) and liver fibrosis.

13-20. (canceled)
